SEQUENCE LISTING (27 sequences)

SEQ ID NO: 1 - DNA, 22 bp, Artificial Sequence
  Synthetic
cgcgtacgcgcaaacagtctga

SEQ ID NO: 2 - DNA, 22 bp, Artificial Sequence
  Synthetic
cgtcgcgaacctgtaaaacagg

SEQ ID NO: 3 - DNA, 22 bp, Artificial Sequence
  Synthetic
cgatatcgatgtactgaatggg

SEQ ID NO: 4 - DNA, 22 bp, Artificial Sequence
  Synthetic
cgcgtaatatatctgggttttc

SEQ ID NO: 5 - DNA, 22 bp, Artificial Sequence
  Synthetic
cgtacgcgcaaacagtctgaaa

SEQ ID NO: 6 - DNA, 22 bp, Artificial Sequence
  Synthetic
cgtcgtcggttcatcataaatt

SEQ ID NO: 7 - DNA, 22 bp, Artificial Sequence
  Synthetic
tatacgcgtaatatatctgggt

SEQ ID NO: 8 - DNA, 22 bp, Artificial Sequence
  Synthetic
gtagcgcgaacaaaatctgaag

SEQ ID NO: 9 - DNA, 22 bp, Artificial Sequence
  Synthetic
cgtcgattgtgtgaatttggac

SEQ ID NO: 10 - DNA, 22 bp, Artificial Sequence
  Synthetic
cggtatcgttgcagtagcgcga

SEQ ID NO: 11 - DNA, 23 bp, Artificial Sequence
  Synthetic
gctgtttgccgacatgaacgcgg

SEQ ID NO: 12 - DNA, 23 bp, Artificial Sequence
  Synthetic
gaccaacgccgacctgtaccggg

SEQ ID NO: 13 - DNA, 23 bp, Artificial Sequence
  Synthetic
ggagagctggtgtcagttggcgg

SEQ ID NO: 14 - DNA, 23 bp, Artificial Sequence
  Synthetic
gcgggggggacgggcatgggtgg

SEQ ID NO: 15 - DNA, 23 bp, Artificial Sequence
  Synthetic
ggcgcggagacggaggagggcgg

SEQ ID NO: 16 - DNA, 23 bp, Artificial Sequence
  Synthetic
gtcgaggtcgtgggggtggtcgg

SEQ ID NO: 17 - DNA, 23 bp, Artificial Sequence
  Synthetic
ggtgggcggcggcccgtcggtgg

SEQ ID NO: 18 - DNA, 23 bp, Artificial Sequence
  Synthetic
gaagacccccacggagaggacgg

SEQ ID NO: 19 - DNA, 23 bp, Artificial Sequence
  Synthetic
gattggttctgggggcacgccgg

SEQ ID NO: 20 - DNA, 23 bp, Artificial Sequence
  Synthetic
gctccggtccgtcctctccgtgg

SEQ ID NO: 21 - DNA, 23 bp, Artificial Sequence
  Synthetic
gcggctctccgccggctcggggg

SEQ ID NO: 22 - DNA, 60 bp, Artificial Sequence
  Synthetic
tgtgtgtgataaggtacgttctctcaaattgagtgataataattgttatcttaatgcagt

SEQ ID NO: 23 - DNA, 60 bp, Artificial Sequence
  Synthetic
acaagttaatggtttaacttctattaaatgggcagataacaactgttatcttgccactgc

SEQ ID NO: 24 - DNA, 61 bp, Artificial Sequence
  Synthetic
tcaagttggttggtttaacttctattaaatgggctgataacaattgttatttgtctagtgt

SEQ ID NO: 25 - DNA, 60 bp, Artificial Sequence
  Synthetic
ggccgtggtacatttggcttggtttcattgctgggcttgttgccttagctctatgcgtct

SEQ ID NO: 26 - DNA, 57 bp, Artificial Sequence
  Synthetic
ggccgtggtacatttggctaggttttatagctggcttgattgccatagtaatggtga

SEQ ID NO: 27 - DNA, 57 bp, Artificial Sequence
  Synthetic
ggccttggtatgtttggctcggcttcattgctggactaattgccatcgtcatggtta

(19) United States
(12) Patent Application Publication
(10) Pub. No.: US 2021/0324414 A1
(43) Pub. Date: Oct. 21, 2021

(54) COMPOSITIONS AND METHODS FOR SEQUESTERING VIRUSES

(71) Applicants: Massachusetts Institute of Technology, Cambridge, MA (US); University of Massachusetts, Boston, MA (US)

(72) Inventors: Ron Weiss, Newton, MA (US); Jin Huh, Watertown, MA (US); Yingzhong Li, Quincy, MA (US); Eric Goldstein, Cambridge, MA (US); Hava Siegelmann, Amherst, MA (US)

(73) Assignees: Massachusetts Institute of Technology, Cambridge, MA (US); University of Massachusetts, Boston, MA (US)

(21) Appl. No.: 17/232,676

(22) Filed: Apr. 16, 2021

Related U.S. Application Data
(60) Provisional application No. 63/011,071, filed on Apr. 16, 2020.

(51) Int. Cl. C12N 15/86; C07K 16/10; C12N 15/113; C12N 15/11; C12N 9/22; A61K 9/51; A61K 35/28; A61P 31/14; C12N 15/10

(52) U.S. Cl. CPC C12N 15/86; C12N 2710/16643; C12N 15/1137; C12N 15/11; C12N 9/22; A61K 9/5123; A61K 35/28; A61P 31/14; C12N 15/1093; C12N 2310/14; C12N 2320/31; C12N 2320/32; C12N 2310/141; C12N 2310/20; C12N 2800/80; C07K 16/10

(57) ABSTRACT

This disclosure provides engineered genetic systems for sequestering and/or destroying viruses, compositions and cells comprising the genetic systems, and methods of treating viral infections, reducing viral load, and/or reducing viral spread. The disclosure also provides libraries comprising elements to be incorporated into the engineered genetic systems.

### RELATED APPLICATION

This application claims priority under 35 U.S.C. § 119(e) to U.S. Provisional Application Ser. No. 63/011,071, filed Apr. 16, 2020, the entire contents of which are incorporated by reference herein.

### FEDERALLY SPONSORED RESEARCH

This invention was made with Government support under Grant No. RO1 CA206218 and RO1 EB025854 awarded by the National Institutes of Health (NIH). The Government has certain rights in the invention.

### REFERENCE TO THE SEQUENCE LISTING

The present application is being filed along with a Sequence Listing in electronic format. The Sequence Listing is provided as a file entitled M0656.70501US02-SEQ.txt created on Apr. 16, 2021, which is 5,339 bytes in size. The information in electronic format of the Sequence Listing is incorporated herein by reference in its entirety.

### FIELD

Engineered genetic systems for sequestering and/or destroying viruses are provided.

### BACKGROUND

The serious impact of the COVID-19 pandemic on lives and society demonstrates the crucial need for rapid responses to counter future viral spreading before reaching pandemic levels. Current responses, such as vaccination and monoclonal antibodies, usually have long development cycles and can be weakened due to viral mutation. Repurposed drugs are often not guaranteed to be effective, and produce marginal results at best (e.g., Remdesivir). Thus, there is a need for new antiviral therapies.

### SUMMARY

The present disclosure provides engineered genetic systems for sequestering and/or destroying viruses, vectors, cells, compositions, and libraries comprising the genetic systems, and methods of use thereof.

Cells comprising the engineered genetic systems described herein, also referred to as “SpongeBots”, bind the virus with high affinity, facilitating viral endocytosis and sequestering the virus. SpongeBots may additionally destroy the viral genome before the virus has a chance to activate or replicate itself, and/or produce one or more anti-inflammatory molecules to attenuate hyperinflammation. Data from the studies described herein demonstrate that SpongeBots protect target cells (e.g., host cells targeted by a virus) from viral infection and reduce viral load and viral spread. The genetic systems, libraries, and methods described herein provide a plug-and-play approach to rapidly engineer new types of customizable antiviral immunity and constitute a radical shift in how viral attacks will be suppressed to protect populations from future viruses and avoid pandemics.

In one aspect, the present disclosure provides an engineered genetic system comprising (i) one or more nucleotide sequences encoding one or more components that bind a virus; and one or more of: (ii) one or more nucleotide sequences encoding one or more inhibitory oligonucleotides targeting the viral genome; and (iii) one or more nucleotide sequences encoding one or more anti-inflammatory molecules.

In some embodiments, the engineered genetic system comprises (i) one or more nucleotide sequences encoding one or more components that bind a virus; and (ii) one or more nucleotide sequences encoding one or more inhibitory oligonucleotides targeting the viral genome.

In some embodiments, the engineered genetic system comprises (i) one or more nucleotide sequences encoding one or more components that bind a virus; and (iii) one or more nucleotide sequences encoding one or more anti-inflammatory molecules.

In some embodiments, the engineered genetic system comprising (i) one or more nucleotide sequences encoding one or more components that bind a virus; (ii) one or more nucleotide sequences encoding one or more inhibitory oligonucleotides targeting the viral genome; and (iii) one or more nucleotide sequences encoding one or more anti-inflammatory molecules.

In some embodiments, the component that binds the virus is a viral receptor or accessory protein. In some embodiments, the viral receptor or accessory protein is ACE2, TMPRSS2, cathepsin B, cathepsin L, nectin-1, DPP4, CD4, LDLR, low density LDLRAD3, CCR5, CXCR4, SR-B1, CD81, claudin-1, occludin, CAR, NPC1, TIM-1, DC-SIGN, L-SIGN, hMGL, TYRO-3, AXL, MER, JAM-A, αvβ3, αvβ5, Gas6, CD21, AChR, EGFR, EFNB2, CD46, SLAMF1, nectin-4, or a CAM.

In some embodiments, the component that binds the virus is an antibody. In some embodiments, the antibody is an IgM antibody.

In some embodiments, the one or more inhibitory oligonucleotides target the RNA-dependent RNA polymerase of the virus, the 5′ UTR region of the viral genome, and/or the 3′ UTR region of the viral genome.

In some embodiments, at least one of the one or more inhibitory oligonucleotides is an interfering RNA. In some embodiments, at least one of the one or more inhibitory oligonucleotides is an miRNA.

In some embodiments, at least one of the one or more inhibitory oligonucleotides is a clustered regularly interspaced short palindromic repeats (CRISPR) guide RNA (gRNA). In some embodiments, the gRNA is a Cas9/gRNA. In some embodiments, the gRNA is a Cas13/gRNA.

In some embodiments, the engineered genetic system further comprises a nucleotide sequence encoding a Cas protein. In some embodiments, the Cas protein is Cas9. In some embodiments, the Cas protein is Cas13.

In some embodiments, the anti-inflammatory molecule is a cytokine. In some embodiments, the cytokine is IL-4, IL-10, IL-11, IL-13, IL-1Rα, TGFβ, or PGE2.

In some embodiments, the anti-inflammatory molecule is an antibody that binds an inflammatory molecule. In some embodiments, the inflammatory molecule is IL-6, IL-6R, IL-1, IL-12, IL-18, IFNγ, GM-CSF, or TNF-α.

In some embodiments, one or more of (i), (ii), and (iii) are present in one or more vectors. In some embodiments, the one or more vectors are viral vectors. In some embodiments, the viral vectors are adeno associated virus (AAV) vectors, Sendai virus vectors, lentiviral vectors, γ-retroviral vectors, or herpes simplex virus type 1 (HSV-1) amplicons. In some embodiments, one or more of (i), (ii), and (iii) are present in or more HSV-1 amplicons.

In some embodiments, one or more of (i), (ii), and (iii) are present in lipid nanoparticles.

In some embodiments, a nucleic acid molecule comprising the nucleotide sequences of (i), a nucleic acid molecule comprising the nucleotide sequences of (ii), or a nucleic acid molecule comprising the nucleotide sequences of (iii) encodes a self-amplifying RNA.

In one aspect, the present disclosure provides an engineered cell comprising an engineered genetic system of the disclosure. In some embodiments, the engineered cell is a eukaryotic cell. In some embodiments, the engineered cell is a mammalian cell. In some embodiments, the engineered cell is a human cell. In some embodiments, the engineered cell is an immune cell. In some embodiments, engineered cell is a stem cell. In some embodiments, the engineered cell is a mesenchymal stem cell.

In one aspect, the present disclosure provides a method of introducing into, or expressing in, a eukaryotic cell (i) one or more components that bind a virus, and one or more of: (ii) one or more inhibitory nucleic acids targeting the viral genome, and (iii) one or more anti-inflammatory molecules. In some embodiments, the disclosure provides a method of introducing into, or expressing in, a eukaryotic cell (i) one or more components that bind a virus, and (ii) one or more inhibitory nucleic acids targeting the viral genome. In some embodiments, the disclosure provides a method of introducing into, or expressing in, a eukaryotic cell (i) one or more components that bind a virus, and (ii) one or more anti-inflammatory molecules. In some embodiments, the present disclosure provides a method of introducing into, or expressing in, a eukaryotic cell (i) one or more components that bind a virus, (ii) one or more inhibitory nucleic acids targeting the viral genome, and (iii) one or more anti-inflammatory molecules. In some embodiments, the eukaryotic cell is an immune cell. In some embodiments, the eukaryotic cell is a stem cell. In some embodiments, the stem cell is a mesenchymal stem cell.

In some embodiments, the method comprises introducing one or more of (i) one or more nucleotide sequences encoding one or more components that bind a virus; (ii) one or more nucleotide sequences encoding one or more inhibitory oligonucleotides targeting the viral genome; and (iii) one or more nucleotide sequences encoding one or more anti-inflammatory molecules into the eukaryotic cell via a viral vector. In some embodiments, the viral vector is an HSV-1 amplicon. In some embodiments, the viral vector is a lentiviral vector.

In one aspect, the present disclosure provides a library of viral vectors (e.g., HSV-1 amplicons or lentiviral vectors) comprising a plurality of the engineered genetic systems of the disclosure.

In one aspect, the present disclosure provides a method of treating or preventing a viral infection in a subject in need thereof, comprising administering to the subject an effective amount of an engineered genetic system or an engineered cell of the disclosure.

In one aspect, the present disclosure provides a method of reducing viral load in a subject in need thereof, comprising administering to the subject an effective amount of an engineered genetic system or an engineered cell of the disclosure.

In one aspect, the present disclosure provides a method of reducing viral spread in a subject in need thereof, comprising administering to the subject an effective amount of an engineered genetic system or an engineered cell of the disclosure.

In some embodiments, the virus is a DNA virus. In some embodiments, the virus is an RNA virus. In some embodiments, the virus is selected from the group consisting of: Adenoviridae, Coronaviridae, Flaviviridae, Filoviridae, Herpesviridae, Hepadnaviridae, Orthomyxoviridae, Paramyxoviridae, Papillomaviridae, Picornaviridae, Polyomaviridae, Poxviridae, Retroviridae, Rhabdoviridae, and Togaviridae. In some embodiments, the virus is selected from the group consisting of: severe acute respiratory syndrome coronavirus (SARS-CoV), SARS-CoV-2, Middle East respiratory syndrome coronavirus (MERS-CoV), influenza virus, dengue virus, zika virus, ebola virus, variola virus, rabies virus, measles virus, human immunodeficiency virus (HIV), Venezuelan equine encephalitis virus (VEEV), Eastern equine encephalitis virus (EEEV), and HSV-1.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1A is an illustration of viral load dynamics with and without SpongeBot administration. During the two weeks following infection, severe tissue damage from inflammation occurs, and ultimately death. Administering SpongeBot cells reduces and keeps viral load below dangerous thresholds, preventing harmful hyperinflammation and providing the innate and adaptive immune systems time to mount an effective defense. FIG. 1B is an illustration of pandemic preparedness, timeline for anti-virus deployment in prevention of future viral pandemics: immediate readiness if an existing pre-made library element matches new viral characteristics, otherwise a ˜12 week process for creating a new custom-tailored solution.

FIG. 2 is a depiction of SpongeBot deployment via HSV-1 amplicon or MSCs into patient and mechanism of action against SARS-CoV-2 (or other) viral particles.

FIG. 3 is an illustration of the history of viral pandemics, suggesting a future of more of these to come.

FIG. 4 is an illustration of the SpongeBot genetic program to destroy SARS-CoV-2 and attenuate a cytokine release storm. (1) Super-Attractor. Overexpression of ACE2 and TMPRSS2 results in efficient sequestration of viral particles. (2) Destroyer. Upon infection of a SpongeBot, SARS-CoV-2 RNA genome is released into the SpongeBot cytoplasm and is quickly destroyed. (3) Attenuator Inflammation is attenuated by secretion of immunomodulatory factors.

FIG. 5A shows high levels of expression with DNA launched replicon (DREP). A comparison of mKate expression from DREP versus CMV as a function of plasmid DNA concentration when transfected into HEK293 is shown. DREP achieves high levels of mKate expression even for very low DNA concentrations, which is the most likely scenario in vivo.

FIG. 5B shows that LNP-replicon encoding IL-12 remodels the tumor microenvironment and eradicates large established tumors. C57B1/6 mice were inoculated with B16F10 tumors and treated with LNP-replicon encoding mCherry, IL12-alb (upright triangles), or IL12-alb-lum (downward triangles) with ˜50 mm² tumors. IL-12 levels assessed at 1/3/7 days post replicon injection. FIGS. 5C and 5D show tumor area and mouse survival, respectively, over time after one intratumoral injection of 10 μg LNP-replicon into ˜50 mm² B16F10 tumors (n=10 mice/group).

FIGS. 6A and 6B show HSV-1 amplicon delivery of gene payload to immune cells in vivo. Percentage (FIG. 6A) and absolute number (FIG. 6B) of mCherry⁺ cells following HSV-1 injection are shown. DREP based delivery resulted in significant gene expression.

FIGS. 7A-7D show preliminary experimental results demonstrating SpongeBot sequestration of HSV-1. (FIG. 7A) qRT-PCR analysis of Nectin-1 levels for several different cell lines. B16F10 cells, which naturally have very low levels of Nectin-1 mRNA, serve as the base cell line for SpongeBot. B16F10/BFP expresses BFP, but is otherwise the same as B16F10, and is used as control in the sequestration experiments. Ym and 4T1 are the target cells. B16F10/Nectin-1 is genetically engineered to overexpress Nectin-1 and serves as SpongeBot. (FIG. 7B) Our measured HSV-1 infection efficiency of the non-engineered cell lines correlates well with their Nectin-1 levels. (FIG. 7C) Ym target cells were co-cultured with various mixtures of B16F10/BFP and B16F10/Nectin-1-GFP cells. Ym cells always comprised 20% of the population, while the remaining 80% of the population were the two B16F10 cell lines. HSV-1 encoding mKate were then added to the media, and the percentages of each cell type (infected by HSV-1 vs. uninfected) were then determined by FACS 18 hours after addition of HSV-1. As shown, Ym infectivity by HSV-1 decreases as the relative concentration of B16F10/Nectin-1-GFP vs. B16F10/BFP cells increases, implying HSV-1 sequestration by SpongeBot. (FIG. 7D) The same general trend is observed for 4T1 cells.

FIG. 8 shows degradation of target Oct4 RNA using miRNA delivered by lentivirus. U6 promoter driving expression of miRNA targeting Oct4 is packaged in lentivirus and delivered to hiPSCs. The ability to dramatically knockdown Oct4 levels using lentivirus delivery of miRNA demonstrates feasibility of the same mechanism to be used against SARS-CoV-2 RNA genome.

FIG. 9 depicts the evaluation of orthogonality of miRNA targets with human transcriptome.

FIGS. 10A and 10B show gene constructs targeting a class of viruses with evolutionarily closely linked genomes. Shown are partial alignments of MERS-CoV, SARS-CoV, SARS-CoV-2 at the proteinase (FIG. 10A) and spike (FIG. 10B) regions. Sequences for Cas13d gRNAs targeting at least two viruses simultaneously are show in bold. (SEQ ID NOS: 22-24 from top to bottom in FIG. 10A and SEQ ID NOS: 25-27 from top to bottom in FIG. 10B).

FIGS. 11A and 11B show conserved sequence analysis. Using 10,000 sequences from NCBI dataset www.ncbi.nlm.nih.gov/sars-cov-2/from 63 countries, conserved sequences of length 22 and above were discovered. (FIG. 11A) Visualization of top 2,000 conserved blocks' coverage of the SARS-CoV-2 genome over the 10,000 sequences, sorted by frequency. (FIG. 11B) Moving 22nt window analysis of conservation across the SARS-CoV-2 genomes.

FIG. 12 shows ranking of possible human cell surface proteins that interact with S protein based on (panel a) likelihood ratio of interaction and (panel b) mutation distance. Panel c shows the number of virus that interact with human cell surface receptor sorted in a descending order. Only interactions that are proven or have a likelihood ratio (LR) of larger than or equal to 200 on P-HIPSTER are considered.

FIGS. 13A-13C show in vitro and in vivo expression of mKate using DREP. (FIG. 13A) Various cells were seeded, infected with virus, and data collected by FACS 24, 48, 72, and 96 hrs post infection. Mean DREP-mKate expression was 30-60 fold higher than CMV-mKate. (FIG. 13B) For in vivo assay, 1×10⁶ 4 T1 breast cancer cells were injected in mammary fat pad of BALB/C mice. 7 days later, 5×10⁶ pfu CMV-mCherry or DREP-mCherry were injected into tumors of five mice per virus. 24 hrs later, tumors were harvested and mCherry intensity was measured by FACS. DREP-mCherry in vivo expression was 18-fold higher than CMV-mCherry. (FIG. 13C) DREP encoded in lentivirus vectors. Lentivirus 1a and 1b co-infect target cells. Lentivirus 1a expresses non-structural proteins (nSPs) driven by SV40 promoter, with WPRE enhancer. miRNA-1 expressed by H1 promoter processes 3′ UTR of Lentivirus 1b transcripts. Lentivirus 1b expresses payloads as DI particles. 51-nt CSE is recognized by non-structural proteins for amplification of RNA replicon. 2A and CM self-cleave during Payload 1 translation. Payload 2 expressed from subgenomic promoter 2 (SGP2).

FIGS. 14A and 14B show optimization of antibody expression from DREP using subgenomic promoters (SGP1/SGP2). (FIG. 14A) Expression of mVenus and mKate using SGP1/SGP2 pairs. (FIG. 14B) Optimized expression of mAb using SGP1/SGP2 indicates that light chain/heavy chain ratio of about 2.5 (SGP30/15) results in highest mAB expression.

FIGS. 15A-15C show small molecule regulation of protein abundance. (FIG. 15A) Mechanism of regulation with TMP. The protein of interest (in this case the firefly luciferase reporter) is fused to DDd (ecDHFR) and targeted for degradation. Presence of TMP stabilizes the fusion protein. (FIG. 15B) In vivo luminescence measurements in mice injected with LNP-encapsulated self-replicating RNA encoding DDd-fLuc. (FIG. 15C) Dose response curve for luminescence measured in BHK-21 cells electroporated with same RNA as in B.

FIGS. 16A and 16B show regulation of secreted protein abundance with TMP. (FIG. 16A) BHK cells electroporated with RNA Replicon encoding IL-2 fused with DDd and grown in media with different TMP levels. On day 2, supernatants were collected and IL-2 levels measured using ELISA. (FIG. 16B) Cells transfected as in FIG. 16A and grown in media+/−TMP for 2 days. Media was removed on day 2 and replaced with fresh media such that some wells retained the same concentration of TMP and some had TMP either added or removed. On day 3, media was replaced with fresh media again, retaining the same concentration of TMP as on day 3. On day 4, supernatant was collected, and IL-2 levels measured.

FIG. 17A shows a TMP-based OFF switch. DDd-L7Ae is unstable in absence of TMP; addition of TMP stabilizes it and allows repression of mVenus translation. FIG. 17B shows a Dox-based ON switch. tetR-DDX6 represses mVenus translation in absence of Dox; addition of Dox prevents binding to target RNA and allows expression of reporter.

FIGS. 18A-18C shows amplification of signal in response to immune activation. (FIG. 18A) The amplification circuit in response to level of IL-6, TNFα, and IFNβ. (FIG. 18B) Response via the JAK/STAT responsive element on different concentrations of TNFα. (FIG. 18C) Response to different concentrations of IFNβ through the interferon sensitive response element (ISRE). Neon Green represents the unamplified response and mKate is the output of the amplification circuit.

FIG. 19 is a demonstration of the SpongeBot concept, showing viral HSV-1 attack on both Ym and SpongeBot cells, and prevention of uptake of virus by the protected target Ym cells.

FIG. 20 is a depiction of the coronavirus lifecycle.

FIGS. 21A-21B illustrate the selection of targets for SARS-COV-2 destruction.

FIG. 22 depicts an assay to test targeted sequence destruction of SARS-CoV-2 using an engineered SARS-CoV-2/mKate2 construct. Destruction of SARS-CoV-2/mKate2 construct with various combinations miRNAs is shown in the right panel.

### DETAILED DESCRIPTION

The present disclosure provides engineered genetic systems that are broadly applicable for treating or preventing viral infections. The present disclosure also provides systems and methods for preparing for the emergence of future pandemics caused by new infectious viral variants. The approach described herein constitutes a radical shift in how viral attacks will be suppressed to protect the population from future viruses and avoid pandemics. The engineered genetic systems of the disclosure provide a programmable platform for performing viral sequestration, viral destruction, and/or attenuation of hyperinflammation. Anticipated and newly discovered viruses and their variants can be readily programmed into the platform for rapid deployment to patients. The programmable platform enables development of libraries for deployment of antiviral therapies within a few weeks.

For many viral pathogens, viral loads grow quickly after infection. During this period, the innate immune system attempts to fight the pathogen, providing time for the adaptive immune system to develop more specific and effective mechanisms to destroy the virus. However, individuals with decreased immune responses are highly vulnerable to worsened outcomes. For COVID-19, for example, the disease ultimately kills due to a delayed, but hyperactive immune response featuring inflammation in the lungs that leads to hypoxemia, reduced oxygen perfusion and ultimately organ failure and death. Reducing viral loads and viral spread in infected individuals would provide time for the adaptive immune response to fully activate, and dramatically increase patient survival.

SpongeBots, or genetically engineered cells comprising the engineered genetic systems described herein, are engineered to express one or more components that bind a virus and promote cell entry (e.g., by endocytosis). In some embodiments, SpongeBots are further engineered to comprise inhibitory oligonucleotides (e.g., interfering RNA or guide RNA sequences) against conserved RNA/DNA viral genome sequences (e.g., sequences that are common among various viral classes), as a means of destroying captured viral RNA/DNA, thereby suppressing viral spread. In some embodiments, SpongeBots are further engineered to produce one or more anti-inflammatory molecules.

SpongeBots sequester infectious viral particles by binding the virus with high affinity and promoting cell entry. Optionally, SpongeBots may destroy infectious viral particles by destroying the viral genome before the virus has a chance to replicate itself. Further optionally, SpongeBots may produce anti-inflammatory molecules to suppress hyperinflammation. When the engineered genetic systems described herein are administered to an infected patient to generate SpongeBots in vivo or when SpongeBots are administered to an infected patient, the cells reduce viral load to below dangerous thresholds and/or prevent harmful inflammation. This reduction in viral load provides the adaptive immune system time to mount an effective defense. The systems and cells described herein are useful in both therapeutic and prophylactic modes and can be broadly used in the population because of their high safety profile and ability to provide an immuno-suppressive function.

The systems and methods described herein further support rapid development and deployment of effective antiviral therapies for current viruses and future variants of viruses and/or newly identified viruses. Development includes creation of an initial library providing premanufactured antiviral therapies. The library is based on known and likely viral cell entry proteins identified via computer analysis prediction, as well as conserved RNA/DNA sequence targets for viral genome destruction (e.g., viral genome sequences that are similar among viral classes). When a new variant or new virus is identified, the library is queried to find entries that match the binding receptor and sequence of the new variant or new virus. Matches provide a pre-existing solution that can be immediately scaled up and deployed to large populations. The speed of response significantly reduces viral reservoir growth and viral mutations. If no library entry fits the new variant or new virus, SpongeBot plug-and-play development tools can still deploy a SpongeBot solution within roughly 12 weeks from identification of a new virus. When viral variants or never-before-seen viruses emerge, SpongeBots are far faster to deploy than vaccines or neutralizing antibodies, with the potential to require only a few weeks from discovery of new virus to deployable therapies.

### I. Engineered Genetic Systems

In some aspects, the disclosure relates to engineered genetic systems comprising (i) one or more nucleotide sequences encoding one or more components that bind a virus (e.g., to facilitate viral cell entry); and one or more of: (ii) one or more nucleotide sequences encoding one or more inhibitory oligonucleotides targeting the viral genome (e.g., to destroy the virus); and (iii) one or more nucleotide sequences encoding one or more anti-inflammatory molecules (e.g., to attenuate inflammation).

In some embodiments, the engineered genetic system comprises (i) one or more nucleotide sequences encoding one or more components that bind a virus; and (ii) one or more nucleotide sequences encoding one or more inhibitory oligonucleotides targeting the viral genome.

In some embodiments, the engineered genetic system comprises (i) one or more nucleotide sequences encoding one or more components that bind a virus; and (iii) one or more nucleotide sequences encoding one or more anti-inflammatory molecules.

In some embodiments, the engineered genetic system comprises (i) one or more nucleotide sequences encoding one or more components that bind a virus; (ii) one or more nucleotide sequences encoding one or more inhibitory oligonucleotides targeting the viral genome; and (iii) one or more nucleotide sequences encoding one or more anti-inflammatory molecules.

The engineered genetic systems of the disclosure comprise engineered nucleic acids (e.g., at least two nucleotides covalently linked together, and in some instances, containing phosphodiester bonds, referred to as a phosphodiester backbone) that do not occur in nature. The engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from one or more sources. A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5 [symbol] exonuclease, the 3 [symbol] extension activity of a DNA polymerase and DNA ligase activity. The 5 [symbol] exonuclease activity chews back the 5 [symbol] end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A. Components that Bind a Virus

The engineered genetic systems described herein comprise one or more nucleotide sequences that encode one or more components that bind a virus. This module is also referred to herein as an “Attractor.” As used herein, a component that binds a virus refers to a virus-specific protein or fragment thereof that binds a virus and/or facilitates cell entry of the virus. When expressed by a SpongeBot, the one or more components that bind a virus bind the virus and promote viral entry into the SpongeBot (e.g., by endocytosis).

The component that binds a virus is capable of binding to at least one viral component. In some embodiments, the viral component is located on the surface of the virus. In some embodiments, the viral component is a structural protein. In some embodiments, the viral component is coronavirus spike protein (S), EEEV E1 or E2, ebolavirus glycoprotein (GP), HIV envelope glycoprotein gp120, or influenza hemagglutinin (HA).

In some embodiments, the component that binds a virus is a viral receptor. In some embodiments, the viral receptor is angiotensin-converting enzyme (ACE2), nectin-1, dipeptidyl-peptidase 4 (DPP4), CD4, low density lipoprotein receptor (LDLR), low density lipoprotein receptor class A domain containing 3 (LDLRAD3), C—C chemokine receptor 5 (CCR5), C—X—C chemokine receptor type 4 (CXCR4), scavenger receptor, class B type 1 (SR-B1), cluster of differentiation 81 (CD81), claudin-1, occludin, coxsackievirus and adenovirus receptor (CAR), Niemann-Pick Cl protein (NPC1), T cell immunoglobulin mucin domain-1 (TIM-1), dendritic cell-specific intercellular adhesion molecule-3-Grabbing on-integrin (DC-SIGN), liver/lymph node-specific intracellular adhesion molecules-3 grabbing non-integrin (L-SIGN), human macrophage lectin specific for galactose/N-acetylgalactosamine (hMGL), TYRO3 protein tyrosine kinase (TYRO-3), AXL receptor tyrosine kinase (AXL), MER receptor tyrosine kinase (MER), junctional adhesion molecule A (JAM-A), αvβ3, αvβ5, growth arrest-specific gene 6 (Gas6), cluster of differentiation 21 (CD21), acetylcholine receptor (AChR), epidermal growth factor receptor (EGFR), ephrin B2 (EFNB2), cluster of differentiation 46 (CD46), signaling lymphocyte activation molecule (SLAMF1), nectin-4, or a cell adhesion molecules (CAM).

In some embodiments, the component that binds a virus is an accessory protein that facilitates cell entry of the virus (e.g., by viral endocytosis). In some embodiments, the accessory protein is a protease. In some embodiments, the accessory protein is TMPRSS2, cathepsin B, or cathepsin L.

In some embodiments, an engineered genetic system of the disclosure comprises one or more nucleic acids encoding ACE2 and/or TMPRSS2. When expressed by SpongeBots, ACE2 and/or TMPRSS2 bind SARS-CoV-2 or SARS-CoV and promote viral entry into the cytoplasm of the SpongeBot. In some embodiments, an engineered genetic system of the disclosure comprises one or more nucleic acids encoding DPP4. When expressed by SpongeBots, DPP4 binds MERS-CoV and promotes viral entry into the cytoplasm of the SpongeBot. In some embodiments, an engineered genetic system of the disclosure comprises one or more nucleic acids encoding Nectin-1. When expressed by SpongeBots, Nectin-1 binds HSV-1 and promotes viral entry into the cytoplasm of the SpongeBot. In some embodiments, an engineered genetic system of the disclosure comprises one or more nucleic acids encoding CD4. When expressed by SpongeBots, CD4 binds HIV and promotes viral entry into the cytoplasm of the SpongeBot. In some embodiments, an engineered genetic system of the disclosure comprises one or more nucleic acids encoding NPC1 and/or TIM-1. When expressed by SpongeBots, NPC1 and/or TIM-1 bind ebolavirus and promote viral entry into the cytoplasm of the SpongeBot. In some embodiments, an engineered genetic system of the disclosure comprises one or more nucleic acids encoding TIM-1. When expressed by SpongeBots, TIM-1 binds EEEV and promotes viral entry into the cytoplasm of the SpongeBot.

A viral receptor or accessory protein also refers to a viral receptor or accessory protein that includes modifications, such as deletions, additions, and substitutions (generally conservative in nature as would be known to a person in the art) to the native sequence, as long as the protein maintains the desired activity relevant to the purposes of the described methods. These modifications can be deliberate, as through site-directed mutagenesis, or can be accidental, such as through mutations of hosts that produce the proteins, or errors due to PCR amplification or other recombinant DNA methods.

The engineered genetic systems of the disclosure may be used to sequester viruses with known receptors as well as viruses with unknown receptors.

In some embodiments, the component that binds a virus is an antibody that is capable of binding specifically to a viral component. In some embodiments, the antibody is an IgM antibody. For viruses with unknown cell entry proteins, an antibody that is capable of specific binding to a viral component can be used. For example, influenza viral cell entry proteins are currently unknown. A high affinity attractor can be designed by fusing the Fab of an antibody against a viral component to Fcα/μ to generate chimeric IgM against it. Chimeric IgM creates a new attractor that sequesters the virus by IgM-mediated endocytosis (32). Similar methods could be applied against future viruses or variants until a cell entry point is discovered.

An antibody is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, and any other modified configuration of the immunoglobulin molecule that comprises an antigen recognition site for the target.

In some embodiments, an engineered genetic system of the disclosure comprises one or more nucleotide sequences that encode a single component (e.g., a viral receptor, accessory protein or antibody) that binds a virus. In some embodiments, an engineered genetic system comprises one or more nucleotide sequences that encode two or more components that bind a virus. For example, in some embodiments, an engineered genetic system comprises one or more nucleotide sequences that encode at least 2, at least 3, at least 4, or at least 5 different components that bind a virus. In some embodiments, an engineered genetic system comprises one or more nucleotide sequences that encode 2, 3, 4, or 5 different components that bind a virus.

B. Inhibitory Oligonucleotides

In some embodiments, the engineered genetic systems described herein comprise one or more nucleotide sequences encoding one or more inhibitory oligonucleotides targeting the viral genome. This module is also referred to herein as a “Destroyer.” When expressed in SpongeBots, the one or more inhibitory oligonucleotides destroy the viral genome upon cell entry, prior to viral activation and replication.

An inhibitory oligonucleotide of the disclosure may target a conserved region of the viral genome. The viral genome may be an RNA genome or a DNA genome. In some embodiments, an inhibitor oligonucleotide of the disclosure targets a region in the 5′ UTR of the viral genome. In some embodiments, an inhibitory oligonucleotide targets a region in the 3′ UTR of the viral genome. In some embodiments, an inhibitory oligonucleotide targets a gene that is critical for viral replication and/or transcription. In some embodiments, an inhibitory oligonucleotides targets the RNA-dependent RNA polymerase of the virus. In some embodiments, the target region has one or more mismatches (e.g., 1, 2, 3, 4, 5, 6, 7, 8, 9, 10, or more) with any sequence of the SpongeBot. In some embodiments, the target region has one or more mismatches (e.g., 1, 2, 3, 4, 5, 6, 7, 8, 9, 10, or more) with any human sequence.

In some embodiments, a target sequence is a predicted target sequence. Methods for predicting target sequences are described in Example 3.

An inhibitory oligonucleotide of the disclosure inhibits a target in the viral genome. The term “inhibits” encompasses complete (100%) inhibition and partial (less than 100%) inhibition, otherwise referred to as reduction. Thus, an inhibitory oligonucleotide may reduce, e.g., target expression, stability, and/or activity by at least 10%, at least 20%, at least 30%, at least 40%, at least 50%, at least 60%, at least 70%, at least 80%, at least 90%, or at least 95%, relative to a control or baseline level. In some embodiments, the control or baseline level is the expression, stability, and/or activity in the absence of the inhibitory oligonucleotide.

In some embodiments, an engineered genetic system of the disclosure comprises a nucleotide sequence encoding an inhibitory oligonucleotide directed to a single target. In some embodiments, an engineered genetic system comprises one or more nucleotide sequences encoding two or more inhibitory oligonucleotides directed to different targets. For example, in some embodiments, an engineered genetic system comprises one or more nucleotide sequences encoding at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different inhibitory oligonucleotides. In some embodiments, an engineered genetic system comprises one or more nucleotide sequences encoding 1-5, 1-10, 1-15, 1-20, 2-5, 2-10, 2-15, 2-20, 3-5, 3-10, 3-15, 2-20, 4-5, 4-10, 2-15, 4-20, 5-10, 5-15, 5-20, 6-10, 6-15, 6-20, 7-10, 7-15, 7-20, 8-10, 8-15, 8-20, 9-10, 9-15, or 9-20 different inhibitory oligonucleotides. In some embodiments, an engineered genetic system comprises one or more nucleotide sequences encoding 2, 3, 4, 5, 6, 7, 8, 9, 10, 11, 12, 13, 14, 15, 16, 17, 18, 19, or 20 different inhibitory oligonucleotides.

In some embodiments, the inhibitory oligonucleotide is an RNA interference (RNAi) molecule. Non-limiting examples of RNAi molecules include small interfering RNAs (siRNAs), microRNAs (miRNAs), and short hairpin RNAs (shRNAs). Methods of identifying interfering RNAs for use in targeting a nucleic acid sequence are known.

In some embodiments, an inhibitory oligonucleotide is an siRNA. siRNAs are typically double-stranded RNA molecules. In some embodiments, each strand of the siRNA is about 15-60, 15-50, 15-40 15-30, 15-25, 19-25, 20-30, or 20-24 nucleotides in length. In some embodiments, each strand of the siRNA is 15, 16, 17, 18, 19, 20, 21, 22, 23, 24, 25, 26, 27, 28, 29, 30, 31, 32, 33, 34, or 35 nucleotides in length. In some embodiments, at least one strand of the siRNA has a 3′ overhang of 1-5 nucleotides (e.g., 1, 2, 3, 4, or 5 nucleotides). In some embodiments, siRNA is chemically synthesized. siRNA can also be generated by cleavage of longer dsRNA (e.g., dsRNA greater than 25 nucleotides in length) with Dicer. These enzymes process the dsRNA into biologically active siRNA. In some embodiments, a dsRNA is at least 50 nucleotides to 100, 200, 300, 400, or 500 nucleotides in length. A dsRNA may have a length of 1000, 1500, 2000, 5000 nucleotides, or longer.

In some embodiments, an inhibitory oligonucleotide is an miRNA. miRNAs are typically single-stranded RNA molecules. In some embodiments, an miRNA is about 15-60, 15-50, 15-20 40 15-30, 15-25, 19-25, 20-30, or 20-24 nucleotides in length. In some embodiments, an miRNA is 15, 16, 17, 18, 19, 20, 21, 22, 23, 24, 25, 26, 27, 28, 29, 30, 31, 32, 33, 34, or 35 nucleotides in length. In some embodiments, the miRNA is a precursor miRNA (e.g., a premiRNA, or a pri-miRNA). In some embodiments, a precursor miRNA is about 50-150, 60-120, 60-100, or 60-70 nucleotides in length. In some embodiments, a precursor miRNA is at least 50 nucleotides to about 100, 200, 300, 400, or 500 nucleotides in length. A precursor miRNA may be as long as 1000, 1500, 2000, 5000 nucleotides in length, or longer. In some embodiments, the viral genome being targeted is an RNA genome and an inhibitory oligonucleotide is an miRNA.

In some embodiments, the inhibitory oligonucleotide is a CRISPR gRNA. As is known in the art, the CRISPR pathway includes two principal components: an RNA-guided nuclease (Cas nuclease) and a guide RNA (gRNA). A gRNA is a short synthetic RNA composed of a scaffold sequence necessary for the Cas nuclease (e.g., Cas9, Cas12, or Cas13) binding and a user-defined nucleotide “spacer” or “targeting” sequence which defines the genomic target to be modified. Thus, one can change the (genomic) target of a Cas nuclease (e.g., Cas9, Cas12, or Cas13) by simply changing the targeting sequence present in the gRNA. In some embodiments, a spacer sequence is at least 15 nt in length, at least 16 nut in length, at least 17 nt in length, at least 18 nt in length, at least 19 nt in length, at least 20 nt in length, at least 21 nt in length, at least 22 nt in length, at least 23 nt in length, at least 24 nt in length, at least 25 nt in length, at least 26 nt in length, at least 27 nt in length, at least 28 nt in length, at least 29 nt in length, at least 30 nt in length, at least 31 nt in length, at least 32 nt in length, at least 33 nt in length, at least 34 nt in length, or at least 35 nt in length. In some embodiments, a spacer sequence is 15-35, 15-30, 15-25, 15-20, 20-35, 20-30, 25-35, 25-30, 18-24, 18-25, 22-30, 22-25, or 22-24 nt in length. In some embodiments, a spacer sequence is 15, 16, 17, 18, 19, 20, 21, 22, 23, 24, 25, 26, 26, 27, 28, 29, 30, 31, 32, 33, 34, or 35 nt in length. In some embodiments, a gRNA has a length of 40 to 100 nucleotides. For example, a gRNA may have a length of 40-90, 40-80, 40-70, 40-60, 40-50, 40-45, 50-60, 50-70, 50-80, or 50-100. Longer gRNAs are encompassed by the present disclosure. Methods of identifying gRNAs for use in modifying or deleting a nucleic acid sequence are known.

In some embodiments, a nucleotide sequence encoding a Cas nuclease is additionally provided. In some embodiments, the Cas nuclease is a Type II enzyme. In some embodiments, the Cas nuclease is a Cas9 nuclease and the guide RNA is a Cas9 guide RNA. In some embodiments, the Cas nuclease is a Type V enzyme. In some embodiments, the Cas nuclease is a Cas12 nuclease (e.g., Cas12a) and the guide RNA is a Cas12 (e.g., Cas12a) guide RNA. In some embodiments, the Cas nuclease is a Type III or Type VI CRISPR enzyme. Type III and Type VI CRISPR enzymes are specialized for targeting RNA. In some embodiments, the Cas nuclease is Cas13 (e.g., Cas13a, Cas13b, Cas13c, or Cas13d) and the gRNA is a Cas13 (e.g., Cas13a, Cas13b, Cas13c, or Cas13d) gRNA. Cas 9 nuclease, Cas12 nuclease, and Cas13 nuclease variants are also encompassed herein.

In some embodiments, the viral genome being targeted is a DNA genome and the Cas nuclease is Cas9 and the gRNA is a Cas9 gRNA. In some embodiments, the viral genome being targeted is an RNA genome and the Cas nuclease is a Cas13 nuclease (e.g., Cas13a, Cas13b, Cas13c, or Cas13d) and the guide RNA is a Cas13 (e.g., Cas13a, Cas13b, Cas13c, or Cas13d) gRNA.

C. Anti-inflammatory Molecules

In some embodiments, the engineered genetic systems of the disclosure comprise one or more nucleotide sequences encoding one or more anti-inflammatory molecules. This module is also referred to herein as an “Attenuator.” When expressed and/or secreted by SpongeBots, the one or more anti-inflammatory molecules attenuate or reduce inflammation.

In some embodiments, the one or more anti-inflammatory molecules attenuate or reduce inflammation by at least 10%, at least 20%, at least 30%, at least 40%, at least 50%, at least 60%, at least 70%, at least 80%, at least 90%, at least 95%, or 100% relative to a control or baseline level. In some embodiments, the control or baseline level is the level of inflammation in the absence of the anti-inflammatory molecule (e.g., in an untreated subject).

In some embodiments, at least one anti-inflammatory molecule is a cytokine. Anti-inflammatory cytokines include, but are not limited to, IL-4, IL-10, IL-11, IL-13, IL-1Rα, TGFβ, or PGE2. In some embodiments, an engineered genetic system of the disclosure comprises one or more nucleotide sequences that encode IL-4 and/or IL-10.

In some embodiments, at least one anti-inflammatory molecule is an antibody that specifically binds to an inflammatory molecule. In some embodiments, the anti-inflammatory molecule is an antibody that specifically binds to IL-6, IL-6R, IL-1, IL-12, IL-18, IFNγ, GM-CSF, or TNF-α. In some embodiments, an engineered genetic system of the disclosure comprises one or more nucleotide sequences that encode an anti-IL-6 antibody and/or an anti-IL-6R antibody. In some embodiments, an antibody that specifically binds to an anti-inflammatory molecule inhibits the activity of the inflammatory molecule by at least 10%, at least 20%, at least 30%, at least 40%, at least 50%, at least 60%, at least 70%, at least 80%, at least 90%, at least 95%, or 100% relative to a control or baseline level. In some embodiments, the control or baseline level is activity of the inflammatory molecule in the absence of the antibody (e.g., in an untreated subject). Antibodies targeting anti-inflammatory molecules are known in the art. Antibodies targeting anti-inflammatory molecules include but are not limited to sarilumab, tocilizumab, siltuximab, infliximab, adalimumab, golimumab, and certolizumab.

In some embodiments, an engineered genetic system of the disclosure comprises one or more nucleotide sequences that encode a single anti-inflammatory agent. In some embodiments, an engineered genetic system comprises one or more nucleotide sequences that encode two or more anti-inflammatory agents. For example, in some embodiments, an engineered genetic system comprises one or more nucleotide sequences that encode at least 2, at least 3, at least 4, or at least 5 anti-inflammatory agents. In some embodiments, an engineered genetic system comprises one or more nucleotide sequences that encode 2, 3, 4, or 5 different anti-inflammatory agents.

In some embodiments, an engineered genetic system of the disclosure comprises one or more nucleotide sequences that encode one or more of IL-4, IL-10, an antibody that specifically binds to IL-6, and an antibody that specifically binds to IL-6R. In some embodiments, an engineered genetic system of the disclosure comprises one or more nucleotide sequences that encode IL-4, IL-10, and an antibody that specifically binds to IL-6. In some embodiments, an engineered genetic system of the disclosure comprises one or more nucleotide sequences that encode IL-4, IL-10, and an antibody that specifically binds to IL-6R.

D. Regulatory Sequences

Expression of the components of the components of the engineered genetic systems of the disclosure may be controlled using one or more regulatory sequences such as enhancers and promoters, operably linked to the nucleotide sequences encoding the components.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. Nos. 4,683,202 and 5,928,906).

A promoter may be a constitutive promoter. Constitutive promoters include any constitutive promoter described herein or known to one of ordinary skill in the art. One example of a suitable promoter is the immediate early cytomegalovirus (CMV) promoter sequence. This promoter sequence is a strong constitutive promoter sequence capable of driving high levels of expression of any nucleotide sequence operably linked thereto. Another example of a suitable promoter is elongation factor-1a (EF-1a). Other constitutive promoters include the simian virus 40 (SV40) early promoter, mouse mammary tumor virus (MMTV) promoter, human immunodeficiency virus (HIV) long terminal repeat (LTR) promoter, Moloney murine leukemia virus (MoMuLV) LTR promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Inducible promoters are also contemplated herein. An “inducible promoter” refers to a promoter that is characterized by regulating (e.g., initiating or activating) transcriptional activity when in the presence of, influenced by or contacted by an inducer signal.

An inducible promoter may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters include any inducible promoter described herein or known to one of ordinary skill in the art. In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters). In some embodiments, an inducible promoter is a metallothionine promoter, a glucocorticoid promoter, a progesterone promoter or tetracycline promoter. In some embodiments, an inducible promoter is responsive to inflammation. In some embodiments, an inducible promoter comprises a cytokine response element (e.g., responsive to the level of IL-6, TNF-α, or TNF-β).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. An enhancer is a short (50-1500 bp) region of DNA that can be bound by activators to increase the likelihood that transcription of a particular gene will occur. Enhancers are cis-acting and can be located up to 1 Mbp (1,000,000 bp) away from the gene, upstream or downstream from the transcription start site. Enhancers are found both in prokaryotes and eukaryotes. There are hundreds of thousands of enhancers in the human genome. The spacing between promoter elements frequently is flexible, so that promoter function is preserved when elements are inverted or moved relative to one another. In the thymidine kinase (tk) promoter, for example, the spacing between promoter elements can be increased to 50 bp apart before activity begins to decline. Depending on the promoter, it appears that individual elements can function either cooperatively or independently to activate transcription.

In some embodiments, a nucleic acid further comprises additional regulatory sequences, including, without limitation, a 3′ untranslated region (3′-UTR), and/or a poly-adenylation (polyA) signal sequence.

In some embodiments, a nucleic acid molecule comprising the nucleotide sequence(s) encoding one or more components that bind a virus, a nucleic acid molecule comprising the nucleotide sequence(s) encoding one or more inhibitory oligonucleotides targeting the viral genome or a nucleic acid molecule comprising the nucleotide sequence(s) encoding one or more anti-inflammatory molecules encodes a self-amplifying RNA. DNA-launched RNA replicons (DREP) are described in International Publication No. WO 2020/181058, the contents of which are incorporated herein by reference. In some embodiments, a DREP is used to express more than one (e.g., 2, 3, 4, 5, or more) transgenes simultaneously. A DREP system can be used to obtain very high levels of transgenes (e.g., a component that binds a virus or an anti-inflammatory molecule). The promoter operably linked to the nucleic acid molecule is used to launch the RNA replicon from the DNA molecule. In some embodiments, the promoter is a constitutive promoter. Any constitutive promoters described herein or known in the art may be used to launch the RNA replicon (e.g., a CMV promoter or a variant thereof). In some embodiments, the constitutive promoter is an enhancer linked to a minimal CMV promoter. In some embodiments, the promoter is an inducible promoter. Any known inducible promoters described herein and are known in the art may be used. When an inducible promoter is used to launch the RNA replicon, an inducer that activates the inducible promoter can be added at a time when transgene expression is desired. The inducible can also be removed when no transgene expression is desired, such that temporary expression of the transgene is achieved.

### II. Vectors

The present disclosure provides vectors comprising the modules of the engineered genetic systems described above. A vector is any nucleic acid that may be used as a vehicle to deliver exogenous (foreign) genetic material to a cell. A vector, in some embodiments, is a DNA sequence that includes an insert (e.g., one or more nucleotide sequences encoding one or more components that binds a virus, one or more inhibitory oligonucleotides, or one or more anti-inflammatory molecules) and a larger sequence that serves as the backbone of the vector. Non-limiting examples of vectors include plasmids, viruses/viral vectors, phagemids, cosmids (comprising a plasmid and Lambda phage cos sequences), and artificial chromosomes, any of which may be used as provided herein. In some embodiments, the vector is a viral vector, such as a viral particle. In some embodiments, the viral vector is an adenovirus, AAV, γ-retrovirus, HSV, lentivirus, or Sendai virus vector. In some embodiments, the viral vector is an HSV-1 amplicon. In some embodiments, the viral vector is a lentiviral vector. In some embodiments, the vector is an RNA-based vector, such as a self-replicating RNA vector.

In some embodiments, one or more of the Attractor, Destroyer, and Attenuator modules are present in one or more vectors. In some embodiments, the Attractor and Destroyer modules are present in one vector. In some embodiments, the Attractor and Attenuator modules are present in one vector. In some embodiments, the Destroyer and Attenuator modules are present in one vector. In some embodiments, the Attractor, Destroyer, and Attenuator modules are present in one vector. In some embodiments, the Attractor and Destroyer are present in two vectors. In some embodiments, the Attractor and Attenuator are present in two vectors. In some embodiments, the Attractor, Destroyer, and Attenuator modules are present in two vectors. In some embodiments, the Attractor, Destroyer, and Attenuator modules are present in three vectors.

In some aspects, the methods described herein comprise expressing the Attractor module and one or both of the Destroyer and Attenuator modules in cells. The vectors provided herein may be used to deliver one or more modules of an engineered genetic system to a subject or to a cell. In some embodiments, the vectors are used to deliver a module of an engineered genetic system to a cell in vivo. In some embodiments, the vectors are used to deliver a module of an engineered genetic system to a cell in vitro.

### III. Cells

The present disclosure provides cells comprising the engineered genetic systems of the disclosure (“SpongeBots”). The cells are transfected with the engineered genetic systems of the disclosure. The term “transfected,” “transformed,” or “transduced” as used herein refers to a process by which exogenous nucleic acid is transferred or introduced into the host cell. A “transfected,” “transformed,” or “transduced” cell refers to a cell which has been transfected, transformed or transduced with exogenous nucleic acid and its progeny. Transfection may be in vitro or in vivo in a subject.

In some embodiments, a cell for use in accordance with the present disclosure is a eukaryotic cell. Any suitable eukaryotic cell may be used in the methods of the disclosure. In some embodiments, the cells are mammalian cells. In some embodiments, the cells are human cells. Examples of cells for use in accordance with the present disclosure include, without limitation, stem cells, sperm, ova, immune cells, kidney cells, lung cells, spleen cells, cardiac cells, gastric cells, intestinal cells, pancreatic cells, muscle cells, bone cells, neural cells, brain cells and epithelial cells. In some embodiments, the cells are primary cells. In some embodiments, the cells are cell lines.

In some embodiments, the stem cells are induced pluripotent stem cells (iPSC). In some embodiments, the stem cells are mesenchymal stem cells (MSCs). In some embodiments, the MSCs are isolated from placenta, peripheral blood, fallopian tube, fetal liver, or lung. In some embodiments, the mesenchymal stem cells are placenta-derived mesenchymal stem cells (PDMSCs).

In some embodiments, the cell is an immune cell. Non-limiting examples of immune cells include dendritic cells, monocytes, macrophages, NKT cells, NK cells, basophils, eosinophils, neutrophils, B cells, T cells (CD4 or CD8), and regulatory T cells. In some embodiments, the immune cells are T cells.

In some embodiments, a cell is transfected with the engineered genetic systems of the disclosure in vivo, e.g., in a subject such as a human subject. In some embodiments, a cell is transfected with the engineered genetic systems of the disclosure in vitro. The engineered genetic systems of the disclosure may be introduced into target cells or subjects using any suitable technique, e.g., by, microinjection, nucleofection, electroporation, lipofection, high pressure spraying, biolystics, calcium phosphate precipitation, and the like. In some embodiments, the engineered genetic systems of the disclosure are introduced via a vector. In some embodiments, the engineered genetic systems of the disclosure are introduced via colloidal dispersion systems, such as macromolecule complexes, nanocapsules, microspheres, beads, and lipid-based systems including oil-in-water emulsions, micelles, mixed micelles, liposomes, and lipid nanoparticles.

### IV. Viruses

The engineered genetic systems of the disclosure can be adapted to sequester and/or destroy any suitable virus. In some embodiments, the virus is a DNA virus. In some embodiments, the virus is an RNA virus. In some embodiments, the virus is selected from the group consisting of: Adenoviridae, Coronaviridae, Flaviviridae, Filoviridae, Herpesviridae, Hepadnaviridae, Orthomyxoviridae, Paramyxoviridae, Papillomaviridae, Picornaviridae, Polyomaviridae, Poxviridae, Retroviridae, Rhabdoviridae, and Togaviridae. In some embodiments, the virus is selected from the group consisting of: SARS-CoV (or SARS-CoV-1), SARS-CoV-2, MERS-CoV, influenza virus, dengue virus, zika virus, ebola virus, variola virus, rabies virus, measles virus, HIV, VEEV, EEEV, and HSV-1.

The engineered genetic systems of the disclosure can also be rapidly adapted for sequestering and/or destroying new viruses or new variants of existing viruses once they are identified. Methods of predicting viral cell entry proteins and targets for genome destruction of new viruses and variants that may emerge in the future are described in Example 3.

### V. Libraries

In some aspects, the present disclosure provides libraries of nucleic acids, vectors, or cells comprising a plurality of one or more modules of the engineered genetic systems of the disclosure.

In some embodiments, the present disclosure provides a library of HSV-1 amplicons comprising a plurality of one or more (one, two or three) modules of the engineered genetic systems of the disclosure.

In some embodiments, the present disclosure provides a library of lentiviral vectors comprising a plurality of one or more (one, two, or there) modules of the engineered genetic systems of the disclosure.

In some embodiments, a library of the disclosure comprises a plurality of conserved viral DNA/RNA sequences. In some embodiments, a library of the disclosure comprises a plurality of sequences encoding human cell surface receptors. For example, the “Cell surface protein atlas” contains dataset of −1492 human glycoproteins.

Methods of predicting viral cell entry proteins and targets for genome destruction of new viruses and variants that may emerge in the future are described in Example 3.

The generation of engineered genetic systems and cells for therapeutic use can start immediately after identification of new virus. In case a completely novel virus emerges, the platform supports a rapid development, manufacturing, and deployment cycle, possibly 12-16 weeks until the start of manufacturing, which is significantly faster than antibodies and vaccines for a new virus.

### VI. Methods of Use

In some aspects, the present disclosure provides methods of treating or preventing a viral infection in a subject in need thereof. In some embodiments, the present disclosure provides a method of treating or preventing a viral infection in a subject in need thereof, comprising administering to the subject an effective amount of an engineered genetic system of the disclosure. In some embodiments, the present disclosure provides a method of treating or preventing a viral infection in a subject in need thereof, comprising administering to the subject an effective amount of an engineered cell comprising the engineered genetic system of the disclosure (i.e., a SpongeBot). The engineered genetic systems and cells of the disclosure can be administered prophylactically to subjects who may have been exposed to an infectious virus and or a subject with a high risk of exposure to an infectious agent (e.g., health care workers or family members of an infected subject).

In some aspects, the present disclosure provides methods of reducing a viral load in a subject in need thereof. In some embodiments, the present disclosure provides a method of reducing a viral load in a subject in need thereof, comprising administering to the subject an effective amount of an engineered genetic system of the disclosure. In some embodiments, the present disclosure provides a method of reducing a viral load in a subject in need thereof, comprising administering to the subject an effective amount of an engineered cell comprising the engineered genetic system of the disclosure (i.e., a SpongeBot). In some embodiments, reducing viral load comprises reducing viral titer by at least by at least 10%, at least 20%, at least 30%, at least 40%, at least 50%, at least 60%, at least 70%, at least 80%, at least 90%, at least 95%, or 100% relative to the viral titer prior to treatment.

In some aspects, the present disclosure provides methods of reducing viral spread in a subject in need thereof. In some embodiments, the present disclosure provides a method of reducing viral spread in a subject in need thereof, comprising administering to the subject an effective amount of an engineered genetic system of the disclosure. In some embodiments, the present disclosure provides a method of reducing viral spread in a subject in need thereof, comprising administering to the subject an effective amount of an engineered cell comprising the engineered genetic system of the disclosure (i.e., a SpongeBot). In some embodiments, reducing viral spread comprises reducing viral spread by at least by at least 10%, at least 20%, at least 30%, at least 40%, at least 50%, at least 60%, at least 70%, at least 80%, at least 90%, at least 95%, or 100% relative to the viral spread prior to treatment.

The present disclosure thus contemplates methods of expressing an engineered genetic system of the disclosure in a subject for treating a viral infection, the method comprising administering to a subject in need thereof an effective amount of an engineered genetic system of the disclosure or an engineered cell comprising an engineered genetic system of the disclosure (i.e., a SpongeBot).

The genetic systems of the disclosure can be adapted for treating any viral infection of interest and/or reducing viral load or viral spread of any virus of interest. In some embodiments, the virus is selected from the group consisting of: Adenoviridae, Coronaviridae, Flaviviridae, Filoviridae, Herpesviridae, Hepadnaviridae, Orthomyxoviridae, Paramyxoviridae, Papillomaviridae, Picornaviridae, Polyomaviridae, Poxviridae, Retroviridae, Rhabdoviridae, and Togaviridae. In some embodiments, the virus is selected from the group consisting of: SARS-CoV, SARS-CoV-2, MERS-CoV, influenza virus, dengue virus, zika virus, ebola virus, variola virus, rabies virus, measles virus, HIV, VEEV, EEEV, and HSV-1.

In some embodiments, a viral infection is a coronavirus infection. Coronaviruses (CoV) are a large family of zoonotic viruses that are transmitted between animals and people, causing illness ranging from the common cold to more severe diseases such as Middle East Respiratory Syndrome (MERS), Severe Acute Respiratory Syndrome (SARS), and COVID-19. A coronavirus infection as used herein may be caused by any coronavirus. In some embodiments, the coronavirus is SARS-CoV, SARS-CoV-2, or MERS-CoV. Several known coronaviruses are circulating in animals that have not yet infected humans. Common signs of coronavirus infection include respiratory symptoms, fever, cough, shortness of breath, and breathing difficulties. In more severe cases, infection can cause pneumonia, severe acute respiratory syndrome, kidney failure, and even death. In some embodiments, the engineered genetic systems and cells provided herein are used to treat a coronavirus infection. In some embodiments, the engineered genetic systems and cells provided herein are used to reduce viral load in a subject with a coronavirus infection. In some embodiments, the engineered genetic systems and cells provided herein are used to reduce viral spread in a subject with a coronavirus infection.

In some embodiments, the present disclosure provides a method of treating or preventing a coronavirus infection, reducing viral load, or reducing viral spread, by administering lipid nanoparticles comprising an engineered genetic system of the disclosure. In some embodiments, the present disclosure provides a method of treating or preventing a coronavirus infection, reducing viral load, or reducing viral spread, by administering one or more vectors comprising an engineered system of the disclosure. In some embodiments, the vector is a viral vector. In some embodiments, the vector is an HSV-1 amplicon. In some embodiments, the present disclosure provides a method for treating or preventing a coronavirus infection, reducing viral load, or reducing viral spread, by administering cells comprising an engineered genetic system of the disclosure (i.e., SpongeBots). In some embodiments, the SpongeBots are immune cells. In some embodiments, the SpongeBots are stem cells. In some embodiments, the SpongeBots are MSCs. In some embodiments, the SpongeBots are PDMSCs.

In some embodiments, a viral infection is an influenza virus infection. An influenza virus infection as used herein may be caused by any strain of influenza virus. In some embodiments, the influenza virus is an influenza type A virus, an influenza type B virus, or an influenza type C virus. In some embodiments, an influenza A strain is selected from the following subtypes: H1N1, H1N2, H1N3, H1N8, H1N9, H2N2, H2N3, H2N8, H3N1, H3N2, H3N8, H4N2, H4N4, H4N6, H4N8, H5N1, H5N2, H5N3, H5N6, H5N8, H5N9, H6N1, H6N2, H6N4, H6N5, H6N6, H6N8, H7N1, H7N2, H7N3, H7N7, H7N8, H7N9, H8N4, H9N1, H9N2, H9N5, H9N8, H10N3, H10N4, H10N7, H10N8, H10N9, H11N2, H11N6, H11N9, H12N1, H12N3, H12N5, H13N6, H13N8, H14N5, H15N2, H15N8, H16N3, H17N10, and H18N11. In some embodiments, the strain of influenza virus is an influenza A (H1N1) strain. In some embodiments, the strain of influenza virus is an influenza A (H5N1) strain. In some embodiments, the engineered genetic systems and cells provided herein are used to treat an influenza virus infection. In some embodiments, the engineered genetic systems and cells provided herein are used to reduce viral load in a subject with an influenza virus infection. In some embodiments, the engineered genetic systems and cells provided herein are used to reduce viral spread in a subject with an influenza virus infection.

In some embodiments, the present disclosure provides a method of treating or preventing an influenza virus infection, reducing viral load, or reducing viral spread, by administering lipid nanoparticles comprising an engineered genetic system of the disclosure. In some embodiments, the present disclosure provides a method of treating or preventing an influenza virus infection, reducing viral load, or reducing viral spread, by administering one or more vectors comprising an engineered system of the disclosure. In some embodiments, the vector is a viral vector. In some embodiments, the vector is an HSV-1 amplicon. In some embodiments, the present disclosure provides a method for treating or preventing an influenza virus infection, reducing viral load, or reducing viral spread, by administering cells comprising an engineered genetic system of the disclosure (i.e., SpongeBots). In some embodiments, the SpongeBots are immune cells. In some embodiments, the SpongeBots are stem cells. In some embodiments, the SpongeBots are MSCs. In some embodiments, the SpongeBots are PDMSCs.

In some embodiments, a viral infection is an EEEV infection. An EEEV infection as used herein may be caused by any strain of EEEV. EEEV infection can result in a systemic febrile illness or neurologic disease, including meningitis (infection of the membranes that surround the brain and spinal cord) or encephalitis (infection of the brain). In some embodiments, the engineered genetic systems and cells provided herein are used to treat an EEEV infection. In some embodiments, the engineered genetic systems and cells provided herein are used to reduce viral load in a subject with an EEEV infection. In some embodiments, the engineered genetic systems and cells provided herein are used to reduce viral spread in a subject with an EEEV infection.

In some embodiments, the present disclosure provides a method of treating or preventing an EEEV infection, reducing viral load, or reducing viral spread, by administering lipid nanoparticles comprising an engineered genetic system of the disclosure. In some embodiments, the present disclosure provides a method of treating or preventing an EEEV infection, reducing viral load, or reducing viral spread, by administering one or more vectors comprising an engineered system of the disclosure. In some embodiments, the vector is a viral vector. In some embodiments, the vector is an HSV-1 amplicon. In some embodiments, the present disclosure provides a method for treating or preventing an EEEV, reducing viral load, or reducing viral spread, by administering cells comprising an engineered genetic system of the disclosure (i.e., SpongeBots). In some embodiments, the SpongeBots are immune cells. In some embodiments, the SpongeBots are stem cells. In some embodiments, the SpongeBots are MSCs. In some embodiments, the SpongeBots are PDMSCs.

In some embodiments, a viral infection is an ebolavirus infection. An ebolavirus infection as provided herein may be caused by any ebolavirus. In some embodiments, the ebolavirus is Zaire ebolavirus, Sudan ebolavirus, Tai Forest ebolavirus, Bundibugyo ebolavirus, Reston ebolavirus, or Bombali ebolavirus. Primary signs and symptoms of an ebolavirus infection include one or more of: fever, aches and pains, weakness and fatigue, gastrointestinal symptoms, abdominal pain, unexplained hemorrhaging, bleeding, or bruising, red eyes, skin rash, and hiccups. In some embodiments, the engineered genetic systems and cells provided herein are used to treat an ebolavirus infection. In some embodiments, the engineered genetic systems and cells provided herein are used to reduce viral load in a subject with an ebolavirus infection. In some embodiments, the engineered genetic systems and cells provided herein are used to reduce viral spread in a subject with an ebolavirus infection.

In some embodiments, the present disclosure provides a method of treating or preventing an ebolavirus infection, reducing viral load, or reducing viral spread, by administering lipid nanoparticles comprising an engineered genetic system of the disclosure. In some embodiments, the present disclosure provides a method of treating or preventing an ebolavirus infection, reducing viral load, or reducing viral spread, by administering one or more vectors comprising an engineered system of the disclosure. In some embodiments, the vector is a viral vector. In some embodiments, the vector is an HSV-1 amplicon. In some embodiments, the present disclosure provides a method for treating or preventing an ebolavirus infection, reducing viral load, or reducing viral spread, by administering cells comprising an engineered genetic system of the disclosure (i.e., SpongeBots). In some embodiments, the SpongeBots are immune cells. In some embodiments, the SpongeBots are stem cells. In some embodiments, the SpongeBots are MSCs. In some embodiments, the SpongeBots are PDMSCs.

In some embodiments, a viral infection is an HSV infection. An HSV infection as used herein may be caused by any HSV. In some embodiments, the HSV virus is HSV-1. In some embodiments, the HSV virus is HSV-2. In some embodiments, the engineered genetic systems and cells provided herein are used to treat an HSV infection. In some embodiments, the engineered genetic systems and cells provided herein are used to reduce viral load in a subject with an HSV infection. In some embodiments, the engineered genetic systems and cells provided herein are used to reduce viral spread in a subject with an HSV infection.

In some embodiments, the present disclosure provides a method of treating or preventing an HSV infection, reducing viral load, or reducing viral spread, by administering lipid nanoparticles comprising an engineered genetic system of the disclosure. In some embodiments, the present disclosure provides a method of treating or preventing an HSV infection, reducing viral load, or reducing viral spread, by administering one or more vectors comprising an engineered system of the disclosure. In some embodiments, the vector is a viral vector. In some embodiments, the vector is an HSV-1 amplicon. In some embodiments, the present disclosure provides a method for treating or preventing an HSV infection, reducing viral load, or reducing viral spread, by administering cells comprising an engineered genetic system of the disclosure (i.e., SpongeBots). In some embodiments, the SpongeBots are immune cells. In some embodiments, the SpongeBots are stem cells. In some embodiments, the SpongeBots are MSCs. In some embodiments, the SpongeBots are PDMSCs.

The terms “subject,” and “patient,” are used interchangeably herein. In some embodiments, a subject is a human subject. In other embodiments, the subject is a livestock animal. The livestock animal may be, for example, a cow, a sheep, a goat, a poultry, or a pig. Other non-human mammals subject to viral infections are also contemplated herein.

An “effective amount” of the compositions of the disclosure generally refers to an amount sufficient to elicit the desired biological response, e.g., express the components of the engineered genetic system in a target cell, treat the condition, etc. In some embodiments, an effective amount is an amount required to prevent viral infection in a subject. In some embodiments, an effective amount is an amount required to treat viral infection in a subject. In some embodiments, an effective amount is an amount required to reduce viral load in a subject. In some embodiments, an effective amount is an amount required to reduce viral spread in a subject. As will be appreciated by those of ordinary skill in this art, the effective amount of an agent described herein may vary depending on such factors as the condition being treated, the mode of administration, and the age, body composition, and health of the subject. Suitable dosage ranges are readily determinable by one skilled in the art.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

### VII. Compositions

In some aspects, the present disclosure provides compositions comprising the engineered genetic systems, vectors, or cells disclosed herein. In some embodiments, the composition is a pharmaceutical composition. In some embodiments, the composition further comprises additional agents (e.g., for specific delivery, increasing half-life, or other therapeutic agents). In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Engineered nucleic acids, in some embodiments, may be formulated in a delivery vehicle. Non-limiting examples of delivery vehicles include nanoparticles, such as nanocapsules and nanospheres. See, e.g., Sing, R et al. Exp Mol Pathol. 2009; 86(3):215-223. A nanocapsule is often comprised of a polymeric shell encapsulating an agent. Nanospheres are often comprised of a solid polymeric matrix throughout which the agent is dispersed. In some embodiments, the nanoparticle is a lipid particle, such as a liposome. See, e.g., Puri, A et al. Crit Rev Ther Drug Carrier Syst. 2009; 26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the engineered nucleic acids disclosed herein may be found, for example, in Remington's Pharmaceutical Sciences, 18th edition, Mack Publishing Co., Easton, Pa. (1990) (incorporated herein by reference in its entirety).

For administration to a subject, the engineered genetic systems of the disclosure, or vectors or cells comprising the engineered genetic systems may be formulated in a composition.

### VIII. Methods of Delivery

Any of the engineered genetic systems, vectors, cells, or compositions disclosed herein may be administered to a subject to treat or prevent a viral infection, reduce viral load, or reduce viral spread.

Suitable routes of administration include, without limitation, intravenous, intranasal, intramuscular, intrathecal, or subcutaneous. In some embodiments, an engineered genetic system, vector, cell, or composition of the disclosure is administered intravenously, subcutaneously, intramuscularly intrathecally or intranasally. In some embodiments, an engineered genetic system, vector, cell, or composition of the disclosure is administered intravenously. In some embodiments, an engineered genetic system, vector, cell, or composition of the disclosure is administered intranasally. In some embodiments, an engineered genetic system, vector, cell, or composition of the disclosure is delivered to the lung. Other routes of administration are contemplated herein. The administration route can be changed depending on a number of factors, including the virus or site of infection.

### Examples

### Example 1: Introduction to Examples 2-4

The recent COVID-19 outbreak has taken a huge toll on mortality and morbidity and has proved economically devastating. It has also highlighted a gaping preparedness hole in our ability to prevent and treat previously unseen pathogens. The SARS-CoV-2 virus, which causes the COVID-19 disease, has shown itself to be more communicable than the flu, more virulent and more likely to cause death. It is evident now, that new, never before encountered viruses may show up at any time. Protracted vaccine and effective therapy development times for each new strain or novel virus are wholly insufficient. Vaccination and antibody neutralization approaches have been rushed from five year to one-to-two-year development timelines; however, mutations to the SARS-CoV-2 Spike protein and the emergence of related coronaviruses are a major source of concern that may weaken the intended purposes of these efforts. Vaccine-induced antibodies that fail to neutralize virus mutants risk enhancing viral spreading via antibody-dependent enhancement (ADE) effects.

Described herein is “SpongeBot,” a bioengineered cell-based therapy solution that incapacitates and destroys viral particles immediately upon administration—thereby providing critical support for the body's immune system. Genetically engineered SpongeBot cells, administered systemically or locally: (i) possess cell membranes with overexpressed cell surface receptors that facilitate viral entry (e.g. ACE2 and TMPRSS2 for SARS-CoV-2)—attracting viral particles to SpongeBots while diverting viruses from their normal targets (epithelial cells in the lungs and other organs for SARS-CoV-2), (ii) destroy the viral RNA genome in infected SpongeBot cells via a CRISPR13b/gRNA editing system and/or microRNA degradation, and (iii) attenuate cytokine release syndrome (CRS), a significant source of patient mortality in many viral infections, by secreting immunosuppressive cytokines (IL-10 and IL-4) and/or antibodies against inflammatory cytokines IL-6 (α-IL-6) and IL-6 receptor (α-IL-6R). The objective of this study is to develop a SpongeBot COVID-19 remedy, and concurrently create a SpongeBot library initially for at least three other viruses.

1. Development of SpongeBot SARS-CoV-2 Super-Attractor & Destroyer. SpongeBots are genetically engineered from placenta-derived mesenchymal stem cells (PDMSCs), modifying them to express high levels of: (i) ACE2 viral entry protein and TMPRSS2 protease to attract SARS-CoV-2 viral particles; and (ii) multiple miRNAs targeting conserved gene sequences to destroy SARS-Cov-2. Expression levels of ACE2, TMPRSS2, and miRNA are determined in vitro. Then, in vivo assays are used to measure SpongeBot kinetics in attracting and destroying SARS-CoV-2 virus, as well as SpongeBot biodistribution, cytotoxicity, and persistence in mouse COVID-19 animal models. Finally, therapeutic efficacy and kinetics of SpongeBot Attractor & Destroyer in clearing SARS-CoV-2 infections are tested. As an alternative means of producing SpongeBot cells, HSV-1 amplicon administration to immune cells is also investigated. It has previously been demonstrated that HSV-1 amplicons can be used to deliver genetic payloads for expression in immune cells (including CD4 and CD8 T cells) in vivo.

2. Development of Preparedness Platform for Five Extant Viruses and Future Viral Pandemics. SpongeBots are customized for two classes of viruses: (1) viruses with known entry proteins (e.g., coronavirus, ebola virus, EEEV, etc.), and (2) viruses whose entry protein is unknown but for whom there exist known antibodies (e.g., influenza virus). Library entries that include gRNAs or miRNAs targeting the genomes of these viruses are generated and determine our ability to use conserved sequences for the different viruses. By integrating the fields of virology, and synthetic, cell, system and molecular biology, the SpongeBot platform serves as a major departure from traditional antiviral approaches. SpongeBot solutions contained in the library enable safety testing in clinical trials prior to emergence of new viruses. The library is grown over time, providing an ever-increasing rapid-deployment, anti-viral defensive arsenal. SARS-CoV-2 and related coronaviruses produce viral mutations or unknown viral forms that might not be treatable with existing means, and which require vaccine updates (when technically and logistically feasible). Viral mutations of SARS-CoV-2 and other coronaviruses are predicted through machine learning to determine likely virus/human receptor pairs and other interactions that could enhance SpongeBot and be used in a pandemic preparedness platform. The machine learning methodology utilizes RNA pattern matching, protein-protein interaction studies and databases of viral protein/human receptor matches.

3. Development of SpongeBot Cytokine Release Storm Attenuator to enhance Attractor/Destroyer. Genetic circuits to attenuate hyper-immune responses via secretion of immunosuppressive cytokines IL-4, IL-10, and/or antibodies against inflammatory cytokines IL-6 and IL-6 receptor are developed. Assays to quantify expression in vitro and immunomodulation in vivo are performed. CRS Attenuator genetic elements are combined with SpongeBot Attractor/Destroyer capabilities to create an enhanced SpongeBot.

Significance. The serious impact of the COVID-19 pandemic on lives and society demonstrates the crucial need for rapid responses to counter future viral spreading before these reach pandemic levels. Existing responses, such as vaccination or monoclonal antibodies, are usually slow to develop and can be ineffective due to viral mutation, while repurposed existing drugs are not guaranteed to be effective. The SpongeBot approach constitutes a radical shift in how viral attacks can be suppressed to protect the population from future viruses and avoid pandemics. SpongeBots, which are genetically engineered cells, provide: “defense-in-depth” protection and attack against viral infections by applying multiple layers of safeguards against viral entry into host cells and against viral proliferation within vital tissues; molecular traps that sequester viral particles into SpongeBot cells, thereby clearing viruses from their host; a prophylactic countermeasure, providing both prevention and treatment at various stages of infection; and a plug-and-play approach to rapidly engineer new types of customizable anti-viral immunity.

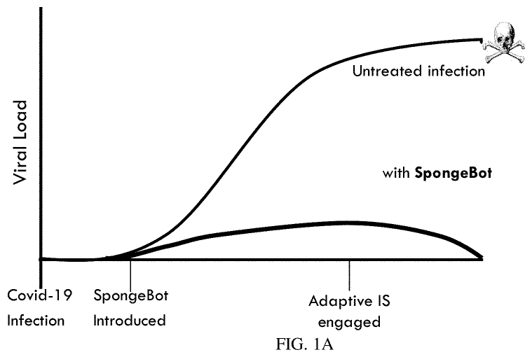

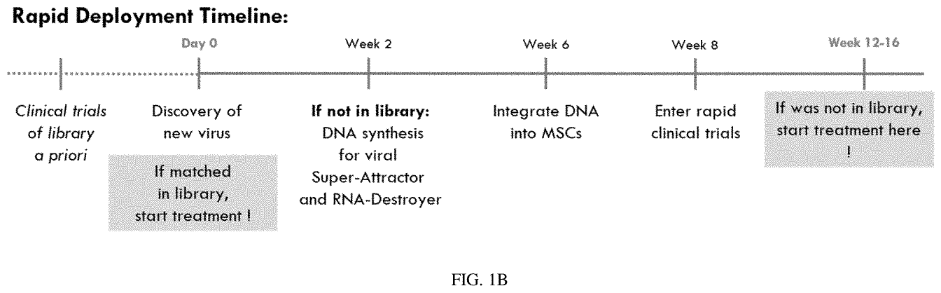

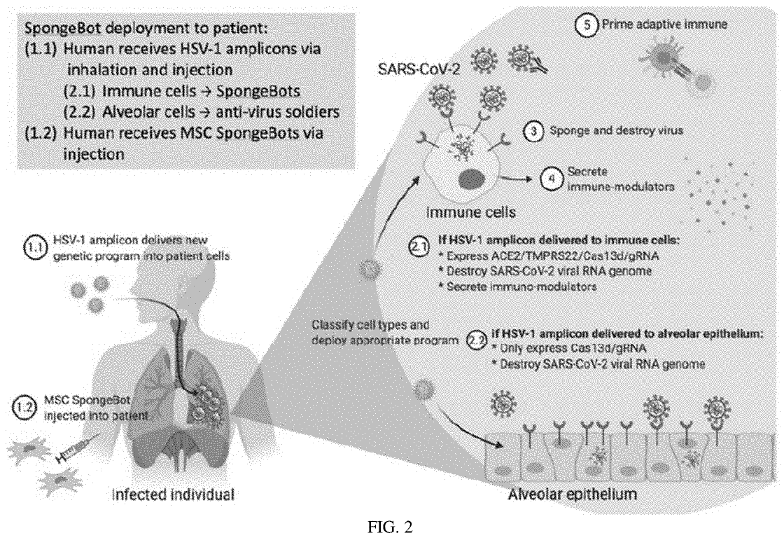

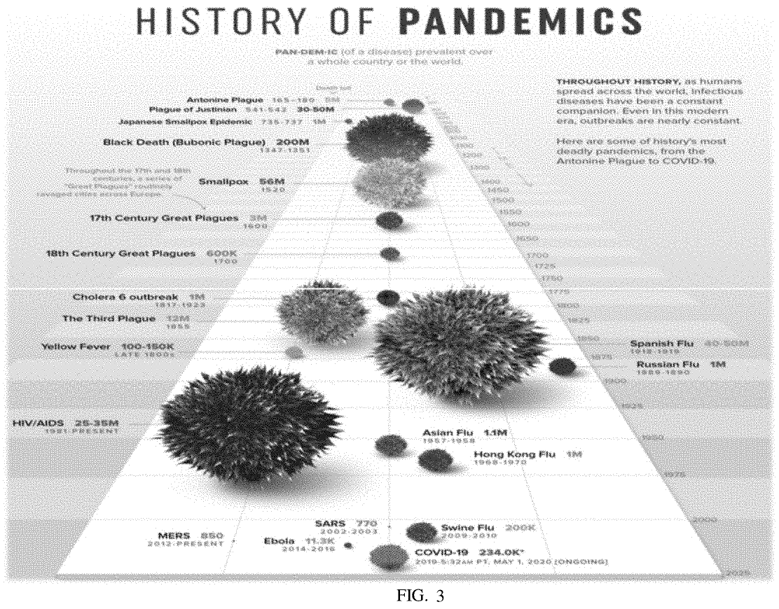

SpongeBots absorb and destroy viral particles near infection sites, thereby providing support to the body's immune system. They are designed to lower viral load (FIG. 1) and/or attenuate hyperinflammation, which is a direct cause of critical illness and death from COVID-19 (1) and other viruses. When virus mutations or never-before-seen viruses emerge, SpongeBots are far faster to deploy than vaccines or neutralizing antibodies, with the potential to require only a few weeks from discovery of new virus to deployable therapies. The studies described herein can prepare a human trial candidate for SARS-CoV-2, and produce SpongeBot variants to target and destroy other viruses, including RNA viruses SARS-CoV-1, MERS-CoV, ebola virus, EEEV, influenza H1N1 and H5N1, and the DNA virus HSV-1 (HSV-1 is also used as a development platform to demonstrate generalizability towards DNA viruses). Mutants and hypothetical future viruses are computationally predicted, and corresponding SpongeBot gene circuits are designed. The SpongeBot plug-and-play genetic circuits are maintained in a library indexed by viral entry proteins and RNA/DNA conserved sequences.

The implementation of SpongeBot is based on two independent but potentially synergistic delivery approaches: genetically engineered mesenchymal stem cells (MSCs) (2) and non-replicating HSV-1 amplicon based in vivo genetic engineering of immune cells. SpongeBots are bioengineered to overexpress entry proteins that viruses use to invade cells, resulting in high affinity viral particle binding to SpongeBot cell surfaces. Once bound to a SpongeBot cell surface, captured viral particles invade the SpongeBot cell. In response, SpongeBot is also genetically engineered to destroy conserved viral RNA/DNA sequences, thereby preventing viral replication and drastically reducing viral load in the body. SpongeBot cells derived from MSCs will tend to migrate via the blood stream to sites of injury, including lung tissue, kidneys, liver and other relevant organs (3,4). MSCs have proven allogeneic safety, as supported by over a thousand MSC clinical trials in the past decade (5), and can be obtained at reasonable cost (described below) (6). SpongeBots based on HSV-1 amplicons provide an alternative means of sequestering and destroying viral particles by in vivo delivery of genetic payload to immune cells (including CD4 and CD8 T cells). Cell type classifier genetic circuits are deployed to ensure that sequestration proteins are only expressed in immune cells, while destruction elements are expressed in all cells. Previous animal studies have demonstrated direct delivery of HSV-1 particles to lung tissue via nasal spray droplets. This serves as a means of delivering the described sequestration and destruction capabilities to cells in infected tissues.

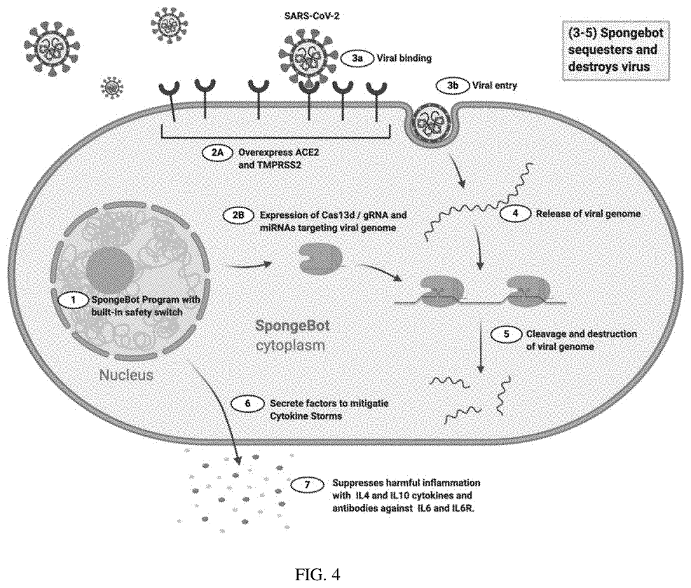

SpongeBot constitutes the core of the proposed pandemic readiness platform. It applies a biologically engineered cell-based therapy that employs multiple anti-viral functions (FIG. 4) to address SARS-CoV-2 and its variants, as well as viruses that will otherwise result in future pandemics. (i) Viral Super-Attractor: SpongeBot overexpresses receptor complexes that bind the virus and promote viral entry (e.g. ACE2 (7) and TMPRSS2 (8) for SARS-CoV-2) into SpongeBot cytoplasm (Example 2; receptors for attracting proteins of other viruses are investigated in Example 3). (ii) Viral RNA Destroyer: Once the virus enters SpongeBot, the customized cell destroys the viral RNA or DNA genome immediately, prior to viral activation and replication (Example 2). (iii) Platform for preparedness to suppress SARS CoV-2 and other viruses: SpongeBots can be programmed with receptors that sequester various types of viruses, including SARS-CoV-2. Additional measures are taken to identify and program receptors to attract viral types with unknown receptor counterparts (e.g., H1N1, H5N1). Finally, future viral attractors and destroyers, resulting from viral mutations or unknown viral forms, are also addressed with the SpongeBot preparedness platform (Example 3). (iv) Cytokine Release Storm (CRS) Attenuator: SpongeBot also attenuates cytokine release storms, thus preventing dangerous, life-threatening inflammation in the lung and other vital organs (Example 4).

SARS-CoV-2 and other viruses are trapped via engineered placenta-derived MSCs (PDMSCs) and/or via HSV-1 amplicon in vivo delivery, and overt immune responses are alleviated by expressing IL-4 (9), IL-10 (10), and/or antibodies against the IL-6 receptor (11). Kinetics of attracting and destroying viral particles and attenuating cytokine storms are determined in existing disease models for moving quickly towards clinical trials.

Choice of PDMSCs and HSV-1 as delivery platforms. In addition to genetically engineered PDMSCs, HSV-1 delivery of genetic circuits is also explored as an alternate, or synergistic option.

MSC features: Safety. MSCs are highly attractive as cell-based therapies due to their well-established allogeneic safety, witnessed by over a thousand MSC clinical trials in the past decade, including 34 approved trials focusing on COVID-19 and FDA permission to use clinically for CODIV-19 compassionate care.

Reasonable cost for scale-up and feasibility of PDMSC-based therapies. Placentas are abundant, pose minimal ethical issues (6, 12), and MSCs derived from them show robust proliferation. Protocols for production of clinical GMP grade PDMSCs are well documented. A single placenta typically yields high quality and high quantity GMP grade PDMSCs (˜4×10⁹) from a 20-liter culture (6) at material cost of ˜$200-400/patient (not including lab costs, medical devices, labor, etc.). This well-documented and understood production process can be scaled up (13).

Genetic engineering. To improve MSC capabilities, preconditioning by stimulants in culture was carried out (14) as well as genetic modifications via adeno-associated virus, lentivirus, retroviruses, Sendai virus, HSV-1, and liposome nucleic acid delivery (15). Using the same DNA delivery vectors, there have been a growing number of pre-clinical studies with genetically engineered MSCs (15). The first genetically engineered MSC in clinical trials used a γ-retroviral vector (16). This study supports the feasibility of using retrovirus vectors (e.g., lentivirus) to deliver the SpongeBot genetic program to MSCs. Accordingly, in some embodiments, the SpongeBot genetic program is packaged in two retroviruses separately and delivered to MSCs via retroviral co-infection. Two clinical studies have tested the safety and efficacy of genetically engineered MSCs in cancer treatments (17, 18). None of these or related studies reported findings of patient risk.

Delivery. MSCs traditionally have been administered via intravenous (IV) injection. Ongoing work is also investigating administering MSCs via aerosols directly to lung tissue, including through a nebulizer (19, 20). The initial focus is on IV delivery of SpongeBots.

Clearance. The reported half-life of MSCs is approximately a day with complete clearance in about 7 days (21). The somewhat rapid in vivo turnover of MSCs is sufficient for therapy (e.g., therapeutic regimen includes two dosages administered four days apart), while enabling SpongeBot clearance even without an OFF switch.

HSV-1 features: Engineered HSV-1 is considered to be a safe viral vector. T-Vec, a modified HSV-1, is FDA approved for clinical use, opening an era of HSV-1 engineering for curing a variety of diseases. Experimental data shows that engineered HSV-1 can infect a variety of cell lines in vitro as well as a number of different immune cells (e.g., T and NK cells) in vivo. Based on these findings, in some embodiments, a non-replicating HSV-1 amplicon is used to transform primary immune cells into SpongeBots for the purpose of attracting and destroying viral particles. An assembly strategy to enable rapid construction and characterization of genetic circuits delivered by HSV-1 amplicon has been previously demonstrated. An important characteristic of HSV-1 is the ability to encode very large payloads (up to 33 kb have been demonstrated). There is also the benefit of known T-Vec production scale-up approaches for transitioning HSV-1 SpongeBot to the clinic.

Platform for engineering genetic circuits up to ˜8 kilobase (kb) in lentivirus. Using a customized MoClo system, genetic parts can be rapidly assembled, including promoters, 5′UTRs, coding sequences, 3′UTRs, enhancers and insulators to create new lentiviruses. An important element in the viral Destroyer lentivirus constructs is the microRNA (miRNA) used to degrade viral RNA genomes. miRNA sensors (22, 23), and lentiviruses that degrade endogenous RNA in pluripotency pathways have been developed previously. A similar approach can be used, here for targeting SARS-CoV-2 and the other RNA genomes.

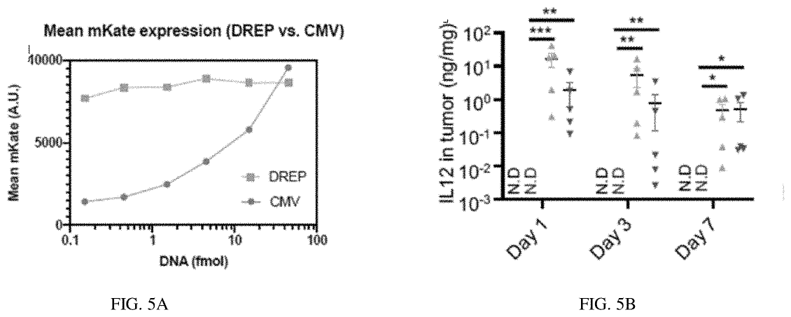

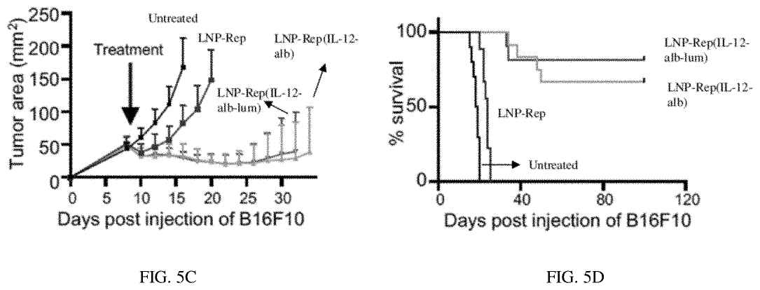

Amplified expression with DNA Launched Replicon (DREP). A platform for obtaining very high levels of gene expression from a single promoter, using self-amplifying RNA (a.k.a. RNA Replicon) has been developed. The replicon is derived from VEEV, but keeps only the non-structural proteins (nsPs), subgenomic promoters, and 3′ UTR elements. Following transcript expression, the non-structural proteins replicate the entire RNA transcript in a self-amplifying fashion that does not depend on further expression of the transcript. In addition, the subgenomic promoter regions within the transcript downstream of the nSPs serve as additional amplification starting points. Experimental results with a DNA launched replicon system, demonstrate a significant increase in expression over the strongest transcriptional promoter (CMV) (FIG. 5A). DNA-launched RNA replicon (DREP) is used to achieve high-level gene expression needed for several SpongeBot elements (e.g., ACE2/TMPRSS2 and/or immunomodulation elements). It has been demonstrated recently that high level expression from a replicon is relevant therapeutically, as a replicon expressing IL-12 and delivered via lipid nanoparticles resulted in significant therapeutic efficacy in a B16F10 large tumor mouse model (24) (FIGS. 5B-5D).

### Example 2. Development of SpongeBot SARS-CoV-2 Super-Attractor & Destroyer

Rationale. This Example focuses on two SpongeBot delivery methods (PDMSCs and HSV-1) against two viruses (HSV-1 and SARS-CoV-2). HSV-1 is used both for delivery of genetic payloads to create SpongeBot, as well as a disease model. This strategy allows the evaluation of a cell-based approach (PDMSC) versus a non-replicating viral particle approach (HSV-1 amplicon) for creating and deploying SpongeBots. The two approaches are likely to have different delivery efficacy, biodistribution, and persistence properties that may turn out to be synergistic. Each approach is studied separately; in some embodiments, a combination approach may be used. Both an RNA virus (SARS-CoV-2) and a DNA virus (HSV-1) are evaluated as a first step to develop and test the platform described herein. SpongeBot viral genome destruction capabilities are effective in both. Both SARS-CoV-2 and HSV-1 are studied in vitro; SARS-CoV-2 is also studied in vivo. HSV-1, an arguably much milder and less urgent disease, serves to quickly prototype the SpongeBot platform system and help determine any unique aspects in targeting a DNA rather than RNA virus (e.g., destroying DNA viral genome with Cas9/gRNA versus destroying RNA genome with miRNA and Cas13d/gRNA).

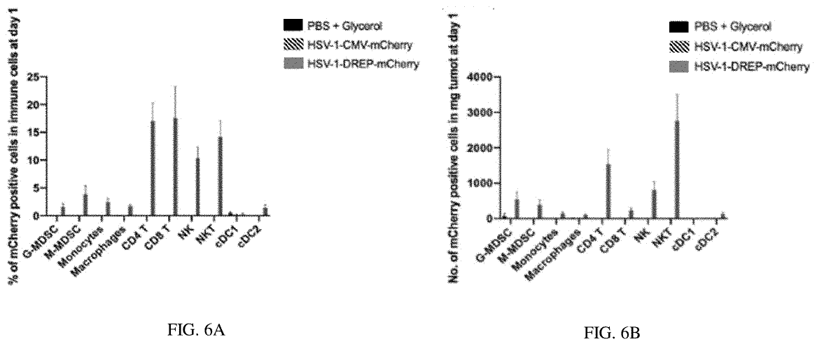

SpongeBot Embodiments. The low immunogenicity and immunomodulatory properties of PDMSCs make them an ideal choice for treating viral infections. PDMSC cells are engineered via lentivirus to express high levels of: (a) viral entry proteins ACE2 and TMPRSS2; and (b) miRNAs targeting conserved regions of SARS-CoV-2. These engineered PDMSCs attract and destroy SARS-CoV-2 in synergy with the host immune system to alleviate viral burden. PDMSCs are easier to access in comparison to fetal liver MSCs, and provide larger quantities of MSCs in comparison to adipose and bone marrow-derived MSCs (12, 25). To deliver SpongeBot payloads to the PDMSCs, second generation self-inactivating lentiviral vectors that are widely used in clinical chimeric antigen receptor (CAR) T cell therapy are used. For initial SpongeBot development, cryopreserved PDMSCs from commercial vendors (e.g., Zen-bio Inc.) are obtained and subsequently clinical grade human PDMSCs isolated from placenta tissue are used. MSC identity is monitored by relevant markers (e.g., CD44⁺, CD166⁺, CD105⁺, CD73⁺, CD90⁺, CD45⁻, CD34⁻, CD14⁻ or CD11b⁻, CD34⁻, CD79a⁻, or CD19⁻, and HLADR⁻) and continuously assayed to detect contaminants (e.g., bacteria and mycoplasma). For HSV-1 delivery, it has recently been shown that an HSV-1 amplicon is able to be delivered to and activate gene expression of various immune cells in a mouse tumor model (FIGS. 6A and 6B). HSV-1 also exhibits low immunogenicity as a delivery platform, and has been used in the clinic. If needed, HSV-1 immunogenicity is further reduced by downregulating innate immune elements via miRNA knockdown.

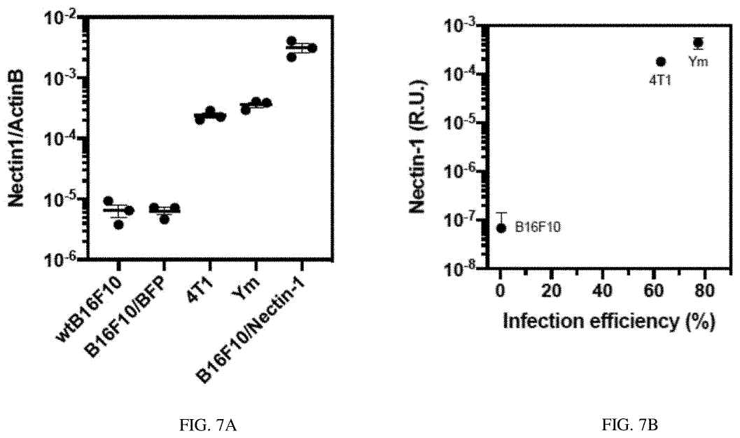

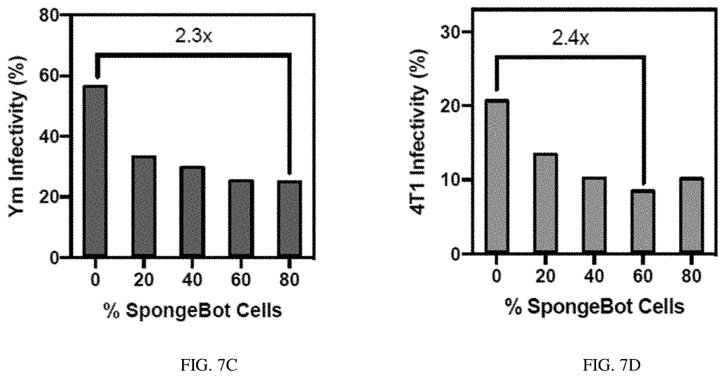

Super-Attractor for HSV-1 and SARS-CoV-2. Cells are genetically engineered to exhibit high HSV-1 and SARS-CoV-2 affinity by over-expressing appropriate cell surface receptors. For HSV-1 sequestration, Nectin-1 is overexpressed, resulting in high binding affinity and infection of engineered cells. FIG. 7 shows preliminary experimental results where B16F10 cells were engineered to over-express mNectin-1. Wildtype Nectin-1 levels for target cells Ym and 4T1 were first measured, and correlated with their HSV-1 infectivity. Cell mixtures containing a fixed amount of Ym or 4T1 cells were co-cultured with various concentrations of B16F10 with or without Nectin-1 expression. As the data shows, increasing the ratio of cells that express Nectin-1 versus those that do not results in a reduction of HSV-1 infection of target cells Ym and 4T1, indicating that B16F10/Nectin-1 SpongeBot cells are able to sequester HSV-1. For SARS-CoV-2 sequestration, ACE2 and TMPRSS2 are overexpressed instead of Nectin. Similar to other efforts described herein, the sequestration performance of genetically engineered PDMSCs and cells engineered via HSV-1 is quantified.

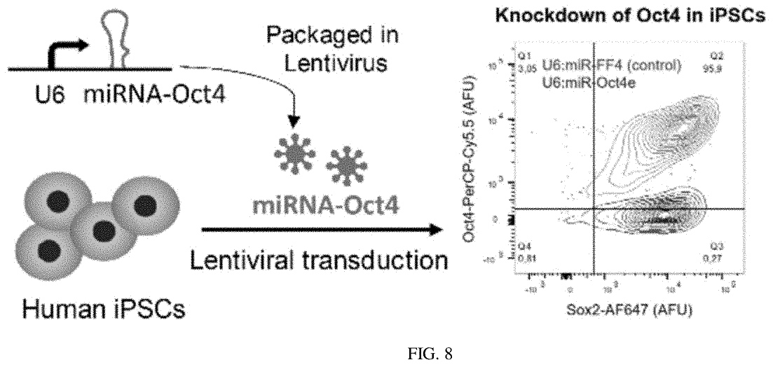

SARS-CoV-2 and HSV-1 Destroyers with miRNA and CRISPR. Efficient degradation of RNA using miRNA expressed from lentivirus has been previously demonstrated in multiple contexts (FIG. 8). Computer-assisted searches are performed to find miRNA sequences that target only the SARS-CoV-2 viral genome and not the SpongeBot transcriptome. In the design, the following are taken into account: (1) target accessibility, (2) potential interactions between the miRNA and the human transcriptome, and (3) conservation in the SARS-CoV-2 genome for potential targets.

Target accessibility: The RISC-miRNA complex does not scan RNAs. Primarily, the interaction between this complex and target RNA happens through Watson Crick base pairing of the miRNA 8nt seed. As a consequence, target RNA must be accessible to the RISC complex, and single stranded. In SARS-CoV-2, transcription of negative strand leads to replication of the 5′ genomic region on all the subgenomic RNAs. The 3′UTR is also replicated on all the subgenomic RNA and, like the 5′ UTR, accessible by the RISC complex. Hence, in some embodiments, miRNAs of the disclosure target the viral 5′ UTR and 3′ UTR regions.

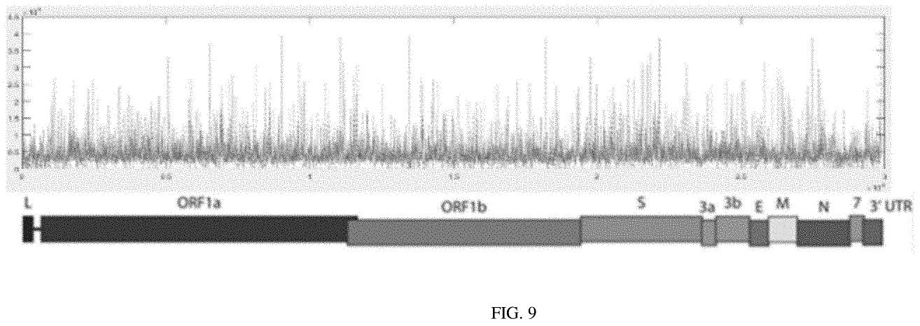

Potential interactions with human transcriptome: In the design of miRNAs against SARS-CoV-2, interactions between the miRNA and endogenous SpongeBot genes must be reduced. Hence, the degree of interaction between the different miRNA designs and the human transcriptome is computationally tested. FIG. 9 shows the computational analysis to estimate the number of human transcriptome hits of SARS-CoV-2 targets. The same analysis can be performed with any other viral genome of interest.

Conserved regions in SARS-CoV-2: It is important to target conserved regions on the viral genome because the miRNA should downregulate/destroy the virus regardless of the particular strain. Both in the 5′ and 3′ UTR, regions that are highly conserved and critical for the virus replication/transcription through RNA-RNA interaction via Watson Crick base pairing were identified; any mutation here will likely be disruptive for the virus and so selected against. A database of different SARS-CoV-2 strains is also used to computationally identify conserved regions.

miRNA design: 70 miRNAs were designed to target conserved regions in the 5′ UTR and the 3′ UTR of the SARS-CoV-2 genome. From this list, the ten that most minimize interactions with the human transcriptome (Table 1) were picked. None of these perfectly match the human transcriptome, so side-effects can be mild.

TABLE 1
miRNA sequences against SARS-CoV-2

| miRNA sequence | SEQ ID NO: | Seed matches |
|---|---|---|
| CGCGTACGCGCAAACAGTCTGA | 1 | 4 |
| CGTCGCGAACCTGTAAAACAGG | 2 | 17 |
| CGATATCGATGTACTGAATGGG | 3 | 18 |
| CGCGTAATATATCTGGGTTTTC | 4 | 19 |
| CGTACGCGCAAACAGTCTGAAA | 5 | 20 |
| CGTCGTCGGTTCATCATAAATT | 6 | 23 |
| TATACGCGTAATATATCTGGGT | 7 | 27 |
| GTAGCGCGAACAAAATCTGAAG | 8 | 30 |
| CGTCGATTGTGTGAATTTGGAC | 9 | 30 |
| CGGTATCGTTGCAGTAGCGCGA | 10 | 31 |

Cas9/gRNA design for HSV-1 destroyer: gRNA sequences against HSV-1 genome were designed based on standard constraints (start with G, include A/T in position 17, and PAM sequence at the end of the 22-nt sequence), and 2,779 candidate sequences were discovered. It was then decided to focus on gRNAs targeting HSV-1 Immediate Early (IE) genes UL48, ICP4, and ICP27, whose disruption should presumably yield the biggest impact, and 27, 80, and 30 candidates, respectively, were found. After performing BLAST against the human genome (HG19), sequences with ≥4 mismatches to any human sequence were chosen, and the candidates closest to the start of the gene coding sequences both on the coding and non-coding/template strands (Table 2) were chosen. The efficacy of lentivirus expressed Cas9/gRNA on HSV-1 ability to replicate after infection of SpongeBot cells is evaluated using plaque assays. Based on measured performance, the set of gRNAs may be expanded, e.g., more sequences for HSV-1 IE and Early genes may be included, as well as sequences that appear multiple times in HSV-1 (there are 14 such candidates). Any off-target Cas9/gRNA effect on SpongeBot cell genomes are quantified. Lastly, the impact of Attractor and Destroyer elements on SpongeBot function is measured both in vitro and in vivo, using viability assays as well as full transcriptome RNA sequencing.

TABLE 2
Candidate gRNA sequences for HSV-1 genome. Right
column is mismatched bases for human matching.

| | SEQ | | Pos | | Human |
|---|---|---|---|---|---|
| | ID | Gene | from | | Genome |
| Sequence | NO: | name | start | Strand | Mismatches |
| GCTGTTTGCCGACATGAACGCGG | 11 | UL48 | 20 | CO | 5 |
| GACCAACGCCGACCTGTACCGGG | 12 | UL48 | 278 | CO | 5 |
| GGAGAGCTGGTGTCAGTTGGCGG | 13 | UL48 | 779 | CO | 6 |
| GCGGGGGGGACGGGCATGGGTGG | 14 | UL48 | 176 | NC | 4 |
| GGCGCGGAGACGGAGGAGGGCGG | 15 | ICP4 | 102 | CO | 4 |
| GTCGAGGTCGTGGGGGTGGTCGG | 16 | ICP4 | 168 | CO | 4 |
| GGTGGGCGGCGGCCCGTCGGTGG | 17 | ICP4 | 63 | NC | 5 |
| GAAGACCCCCACGGAGAGGACGG | 18 | ICP27 | 150 | CO | 4 |
| GATTGGTTCTGGGGGCACGCCGG | 19 | ICP27 | 511 | CO | 6 |
| GCTCCGGTCCGTCCTCTCCGTGG | 20 | ICP27 | 181 | NC | 5 |
| GCGGCTCTCCGCCGGCTCGGGGG | 21 | ICP27 | 93 | NC | 4 |

In vitro Assays to Determine Kinetics of SARS-CoV-2 and HSV-1 Attractors and Destroyers. First, SpongeBot payload expression is determined by qPCR, ELISA, FACS, and Western Blotting. Then, to evaluate Attractor efficiency, engineered PDMSCs and cells engineered with HSV-1 are incubated with and without SARS-CoV-2 and HSV-1. 24 hours later, PDMSCs and SpongeBot PDMSCs are washed and total proteins and RNA are extracted for ELISA and qRT-PCR. To determine Destroyer efficiency, PDMSCs, PDMSCs-Attractor, PDMSCs-Destroyer, and PDMSCs-Attractor-Destroyer are incubated with HSV-1 and SARS-CoV-2 virus. Cells are washed and assayed (viral proteins, RNA) on days 1, 3, 5, 7, and 9 after incubation. Kinetic curves for absorbing and destroying the virus are determined. Based on these kinetic curves, the best time point and assay for live viruses by plaque assay is selected for further kinetic confirmation. Supernatants are used for standard plaque assays with Vero cells to count number of infectious SARS-CoV-2 particles.

In vivo Functional Assays to Determine Efficiency of SARS-CoV-2 Super-Attractor and Destroyer. Human ACE2 expression transgenic mice (26) are purchased (Taconic (27)) and TCID₅₀ of SARS-CoV-2 (SARS-CoV-2/human/USA/WA-CDC-WA1/2020) is used to infect animals via the intranasal route. Briefly, 20 μL of medium containing virus is instilled into each naris using a pipette. Swab, sputum, stool, and blood are collected at 0, 5, 11, and 16 days post infection (dpi) to determine viral load by qPCR and plaque assays. Infected animals are diagnosed by computed tomography (CT) scanning to determine lung damage at 0, 5, 11, 16 dpi; blood routine tests along with ALT and AST are performed. This data helps determine SARS-CoV-2 half lethal dose and desired times for SpongeBot administration. For therapeutic analysis, 10⁶, 3×10⁶, and 10⁷ engineered PDMSCs encoding luciferase gene are delivered into the SARS-CoV-2 infected mice by intravenous or intranasal administration and PDMSC biodistribution is determined, and cytotoxicity and persistence of the PDMSC SpongeBots are also monitored. This data is used to optimize PDMSC SpongeBot dosage and administration route. The SpongeBot HSV-1 therapeutic vector is administered identically to SARS-CoV-2, using 10⁷ PFUs per animal. Infected mice are administrated SpongeBots at SARS-CoV-2 peak day, as well as two days before/after, to mimic clinical settings of early, middle and late-stage virus infection. Mice survival, along with CT scans and routine blood analysis, is quantified to determine therapeutic efficacy.

Alternative Methods. In some embodiments, the proliferation potential of engineered PDMSCs is increased by expressing OCT4, KLF4, SOX2, and GLIS1. In some embodiments, non-signaling mutant versions of receptors (ACE2, TMPRSS2, etc.) are used. In some embodiments, Cas13d/gRNAs is used to target viral genome. In some embodiments, the virus can be destroyed in additional ways, for example, by interfering with nSP activity, or degrading viral proteins directly with programmable proteases. Methods to engineer the translation machinery of SpongeBot to be resistant to the viral shutdown of endogenous translation are also considered. In some embodiments, non-replicating HSV-1 amplicons are administered intranasally to genetically program SpongeBots in vivo. In some embodiments, all engineered cells express miRNA to destroy the SARS-CoV-2 viral genome. In some embodiments, expression of ACE2/TMPRSS2 is restricted to only desired cell types using miRNA-based cell classifiers. With this approach, multi-input miRNA sensing genetic circuits ensure that expression of the cell surface receptors is limited to cells that match a desired miRNA expression profile (e.g., that of CD8 T cells).

Outcomes and Metrics. This study results in the development of the first two versions of SpongeBots comprising SARS-CoV-2 and HSV-1 Super-Attractor and Destroyer. The number of engineered PDMSC SpongeBot cells delivered to human patients is estimated to be ˜10⁸ per patient, while the number of viral particles present in the patient are estimated to be around 10⁹. Hence, SpongeBot cells that bind approximately 5-10 viral particles per day are developed, preventing further viral replication and spread. Virus binding levels are compared between engineered SpongeBot cells and type II pneumocytes to validate the potential of SpongeBot to provide protection to pneumocytes against SARS-CoV-2. Viral DNA and RNA levels are measured by qPCR, comparing SpongeBot Attractor and SpongeBot Attractor/Destroyer cells to estimate replication kinetics and the effect of destroyer modules on reducing replication. Since the aim is to destroy the viral nucleic acid upon entry into SpongeBot cells, effectively preventing measurement of virus levels within cells, the virus absorbed is quantified through measurement of remaining viral particles in the media with plaque assays. The efficiency of virus destruction is also measured by comparing levels of infective viral particles released from Attractor/Destroyer and control cells. Target RNA (SARS-CoV2) and DNA (HSV-1) are degraded at similar rates and viral production is reduced by at least 90%. Viral degradation rates and biodistribution of cells delivered to animals are used to inform therapeutic doses for in vivo treatment of SARS-CoV-2 in animal models, where 75-90% reduction in viral loads are achieved compared to controls without treatment measured in lungs and by swabs 3 days after SpongeBot introduction.

### Example 3. Development of Platform to Address Future Viral Pandemics

Rationale. The SpongeBot solution uniquely lends itself to very rapid development and deployment of effective antiviral therapies for existing and future viruses before they can become pandemics. The platform's library of premanufactured, clinically approved antiviral therapies identified via computer analysis-prediction of likely viral cell entry proteins, as well as conserved RNA/DNA sequence targets for viral genome destruction, allow for unparalleled speed in antiviral production and deployment. When a new virus is identified, the SpongeBot library is queried to find entries that match the new virus binding receptor and sequence. Matches provide a pre-existing solution that can be immediately scaled up and deployed to large populations. The speed of response significantly reduces viral reservoir growth and viral mutations. If no library entry fits the new virus, SpongeBot plug-and-play development tools can still deploy a SpongeBot solution within roughly 12 weeks from identification of a new virus.

Known RNA viruses in addition to SARS-CoV-2. Using Example 2 as a template, SpongeBots are customized to sequester and destroy SARS-CoV, MERS-CoV (38), ebola virus (39), EEEV, and influenza H1N1 and H5N1. The antiviral solutions developed not only address these viruses directly, but also increase the SpongeBot library's known entry proteins and miRNA targeting sequences. For viruses with currently unidentified entry proteins (e.g., influenza H1N1, influenza H5N1), a novel non-neutralizing IgM library is developed to use antibody dependent enhancement (ADE (43)) to trap viruses.

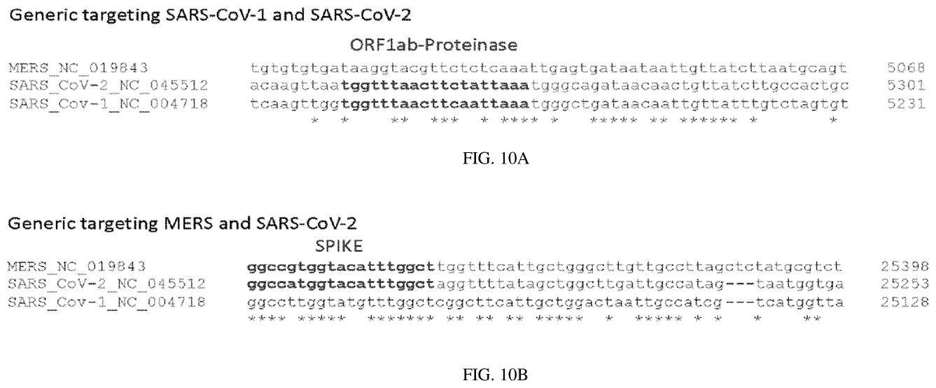

SpongeBot Library: viruses with known binding proteins. The SpongeBot library includes bioengineered SpongeBot cells capable of attracting, sequestering and destroying SARS-CoV, MERS-CoV, ebola virus, and EEEV. Genetically engineered ACE2/TMPRSS2 (8) expressing SpongeBots from Example 1 traps SARS-CoV-1 and MERS-CoV. SpongeBot antiviral cells are engineered to express NPC1 to trap Ebola (28) and Tim-1 to trap EEEV (29). gRNAs and miRNAs to target the RdRP of these viruses separately are also designed (FIG. 10). SARS-CoV, MERS-CoV, and SARS-CoV-2 are beta coronavirus family members with highly conserved genetic information, making it possible to design a generic SpongeBot to destroy viruses in this class. SpongeBots capable of targeting highly conserved sequences common to this viral class can also directly target evolutionarily similar gain-of-function viruses. SpongeBot technology includes the ability to modify gRNA/miRNA sequences allowing SpongeBots to attack viruses at multiple points to increase probability of viral destruction.

SpongeBot Library: viruses with unknown entry proteins. The SpongeBot library can also address viruses with unknown entry proteins. For example, influenza (30) viral entry proteins are currently unknown. For this, the principles of ADE are used to design a high affinity attractor by fusing the Fab of antibodies against the virus (31) to Fcα/μ to generate chimeric IgM against it. Chimeric IgM creates a new attractor that traps the virus by IgM mediated endocytosis (32). Similar methods are applied against future viruses until an entry point is discovered. Since influenza viral genome sequences are known, appropriate gRNAs/miRNAs are designed.

Validating SpongeBot library attractor and destroyer properties. All SpongeBot protein entry receptors and RNA/DNA target sequences included in the library are validated in vitro to check attractor destroyer kinetics by qPCR and plaque assays as in Example 2. Validated SpongeBots are combined with immune modulators from Example 4 for further validation in vivo. All animal models use a clinical scoring system and cage side evaluation to monitor and quantify morbidity; major efficacy evaluation endpoints are noted for each model. Wild type ebola virus does not infect wildtype mice and the effect relates to the interferon response. However, mice are susceptible to mouse adapted EBOV (33) via the intraperitoneal route, which results in a lethal disease and is used in these studies. The major endpoint is survival/non survival. Highly pathogenic influenza virus causes a lethal disease in wildtype mice (34) when infected via the intranasal route, which is used in these studies. The major endpoint is survival/non survival. Wildtype mice are susceptible to EEEV when infected via the intranasal route (35), which is used here. The major endpoint is survival/non survival.

Predicting Viral Mutations for Identification of Conserved Sequences Rationale. Predicting viral variants is an enormous undertaking involving trillions of possible RNA/DNA genomic sequence alterations, making an advanced computational approach the only viable means (36). Single strand RNA viruses, such as SARS-CoV-2, mutate with base substitutions, deletions and insertions at error rates estimated from 10⁻³ to 10⁻⁵ per base per replication cycle (37). By studying the genetic landscape of current SARSCoV-2 using AI neural networks and designing appropriate algorithms, potential viral variants are predicted, to upgrade SpongeBots to counter these threats. The same method is used to predict mutations of additional viruses (those mentioned above and more), enabling the creation of a library of SpongeBot circuits. These SpongeBot variations, once they pass clinical trials, support very rapid development of SpongeBot treatments against newly emerging viruses before they evolve into pandemics.

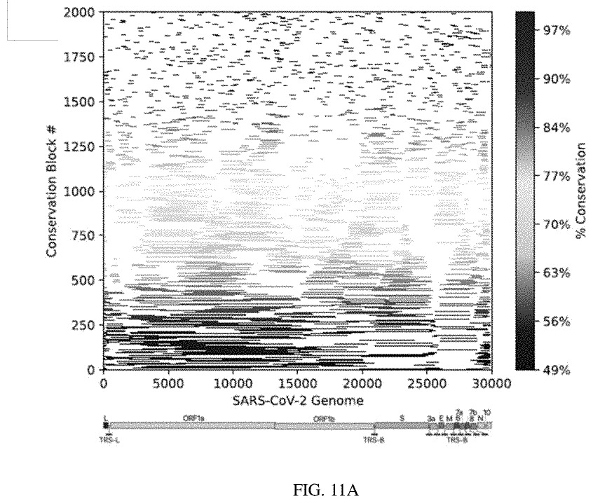

First, large viral genome data repositories were employed to identify conserved sequences within the viral genome, which can be used to destroy the virus. Deep sequencing provides statistics about areas which mutate more frequently than others, common mutation patterns, and assigns entropy to different parts and subsequences of the genome. This information is used to develop a neural machine mutation model for predicting mutations in a semi-supervised manner. The mutation model described herein is based on the GPT-2/3 (Generative Pretrained Transformer) neural network model by Open AI38. The GPT pre-trained language model has demonstrated the ability to generate convincing paragraphs continuations and logically working code when tuned with few examples. The viral genomic sequence is treated as a language and the model is trained to learn it, teaching the system to generate valid sequence variations. SARS-CoV-2 still has a relatively small mutation dataset. To strongly fit the model to valid mutations, the much larger flu NCBI database is also used and other databases are added as needed. The additional datasets provide a secondary supervised learning signal for the model to learn the genomic language of viruses and their mutations. The model avoids overfitting to the small mutation dataset and generalizes better to predicting valid possible future mutations. To ensure safety, viral genetic sequences that are close to human genome substrings are excluded. To ensure feasibility, the adversarial network paradigm is applied: where a discriminator network receives input mutations from the training dataset, a withheld validation dataset, and from the output of the GPT to identify invalid (“made up”) mutations. Propagating this information back to the GPT allows it to tighten its prediction, and the joint learning of these two networks leads to optimal prediction results. FIG. 11 shows early conserved sequence analysis.

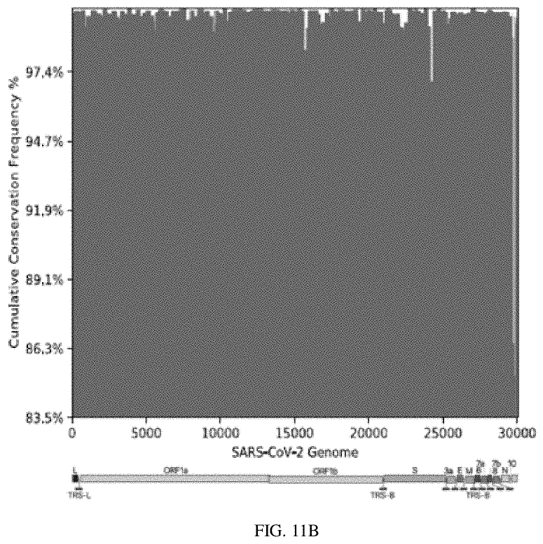

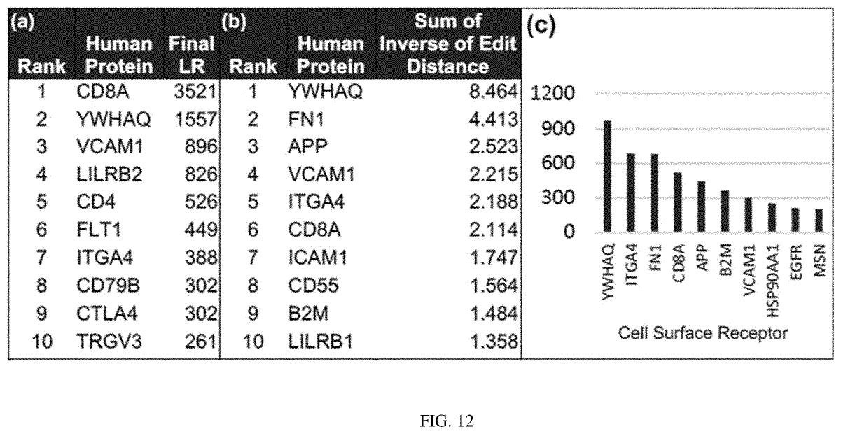

Predicting Future Viral Entry Points. For predicting future viral entry points into human cells, first the P-HIPSTER dataset (39) is used. Possible human cell surface proteins that interact with S protein are ranked. Out of 365 predicted interactions between S protein and human cell surface proteins, 40 human cell surface proteins that are more likely to interact with the S protein ranked by the likelihood ratio of interaction were identified. P-HIPSTER infers the likelihood of these interactions based on sequence- and structure-based information from atomic structures and from homology models. This provides the simplest ranking of human cell surface proteins that may be targeted by S protein, the most likely of them appear in FIG. 12. As a second complementary method, a list of 2,093 human cell surface proteins obtained from EMBL-EBI (40) and Cell Surface Protein Atlas (41) served as the starting point. All host-virus interactions of these human cell surface proteins were identified using P-HIPSTER (39), and only interactions that are either proven or associated with a high likelihood (LR>200 score there) were considered—these corresponded to 485 unique human cell surface proteins. Then interactions associated with S protein were removed and the mutation distance between S protein and virus protein V was computed in the remaining interactions. The edit distance was used to measure the mutation distance, which counts the number of steps (insertion, deleting, and replacement) to jump from one protein sequence to another sequence. D EMBOSS Needle (40) was used to align the two sequences when computing the edit distance. The human cell surface proteins were then ranked by the sum of inverse of edit distance of all its associated interactions, which takes into account both the number of such possible interacting proteins and mutation distance between the virus protein V and S protein in these interactions. Panel (b) of FIG. 12 shows the top human cell surface proteins that appear on the ranking obtained this way. The top 40 human proteins on this ranking have a 37.5% overlapping with the top 40 human proteins on the ranking based on P-HIPSTER's inference that is shown in panel (c) of FIG. 12.

To evaluate the above, viral sequence and viral entry protein predictions are created based on information available in the year 2000 and in the year 2010, and that information is used to predict emergence of viruses during the years 2000-2010 and 2010-2020, respectively. Not just pandemics, but also emergence of seasonal viruses, mutations in existing viruses, and emergence of viruses from the wild that remain fairly localized (an approximately yearly occurrence) are considered.

Alternative Methods. Developing predictive models of viral mutations and future viruses is an important, yet complex undertaking. Though growing, viral entry protein and genome sequence data can be sparse. Two alternative approaches may be applied. In some embodiments, rules about nucleotide change, deletions, and insertions, locations and relations between multiple mutations are identified from deep sequencing databases, and the rules are translated via a neural constructor, into a recurrent neural network, which is then trained on the data. With rules encoded in a network, a far smaller ratio of sequence length to dataset size is required. In some embodiments, co-evolution prediction methods that focus on binding regions are employed.

Outcomes and Metrics. Completion of SpongeBots customized for the known RNA viruses in addition to SARS-CoV-2 described above 2.1 creates a model for multiple viruses and provide a sample library of validated SpongeBots. Sequestration and destruction of viruses with known entry receptors (SARS-CoV, MERS-CoV (4), ebola virus, EEEV) and unknown entry receptors (influenza virus) is evaluated using methods described in Example 2. Similar rates of virus binding (5-10 particles/SpongeBot/day) are achieved for viruses with known entry receptors, and 2-5 particles/SpongeBot/day are achieved for viruses sequestered using ADE. The ability to use IgM to attract viruses (ADE) improves customization of SpongeBot for future pandemics by unknown viruses. Viral production in infected SpongeBot is reduced cells by at least 90% and 75-90% reduction in viral loads is achieved in animal disease models treated with appropriate SpongeBot cells. This study develops the first ever platform that allows antiviral therapy development prior to novel virus discovery and rapid deployment for future viruses shortly after they are identified. The predictive accuracy of the SpongeBot platform increases as the library grows. A prediction accuracy of viral binding to human receptors, including viral mutations, of at least 76% is achieved, matching that of P-HIPSTER.

### Example 4: Programmable Immunosuppressive Microenvironments Using SpongeBot

Rationale. Hyperinflammation is the key factor associated with the ARDS-like pulmonary edema and pulmonary capillary hypercoagulation implicated in the high mortality and serious morbidity of COVID-19 and similar pathogenic diseases. SpongeBot cells are engineered to produce IL-4 and IL-10 to reduce inflammation and attenuate CRS. IL-4 is a cytokine expressed by natural killer T cells (NKT), basophils, mast cells, eosinophils, and is critical for differentiation of Th2 cells to helper B cells. Upon IL-10 expression from monocytes, T helper 2 cells (Th2), mast cells, and CD4 T regulatory cells, an immunosuppressive microenvironment quickly develops and polarization of macrophages to M2 is enhanced (helping tissue repair); IL-10 also upregulates inflammation-suppressive anti-cytokines and down-regulates pro-inflammatory cytokine receptors (43). Co-expression of IL-4 and IL-10 may also repair damaged alveolar sacs and recover the ability of these sacs to exchange oxygen normally. Additionally, studies have shown that blocking IL-6 or IL-6R significantly reduces CRS. Antibodies against IL-6R are approved by FDA for rheumatology as well as for CRS induced by CAR-T cell therapy and by SARS-CoV-2.

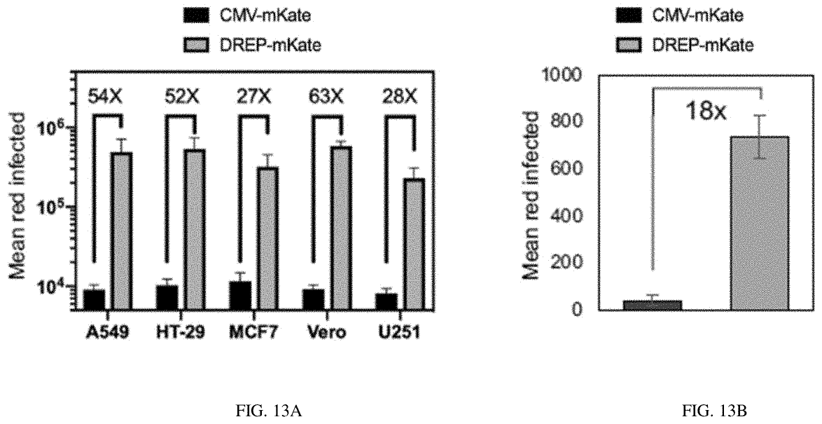

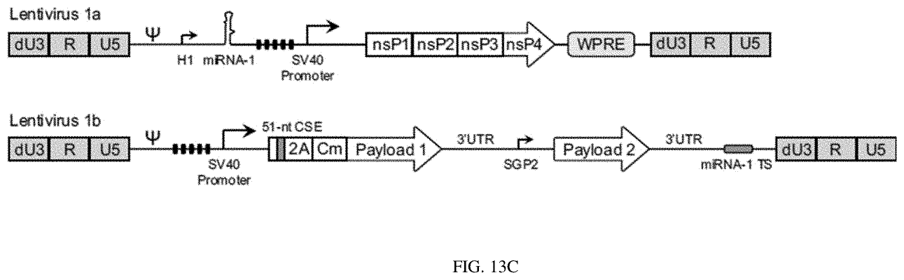

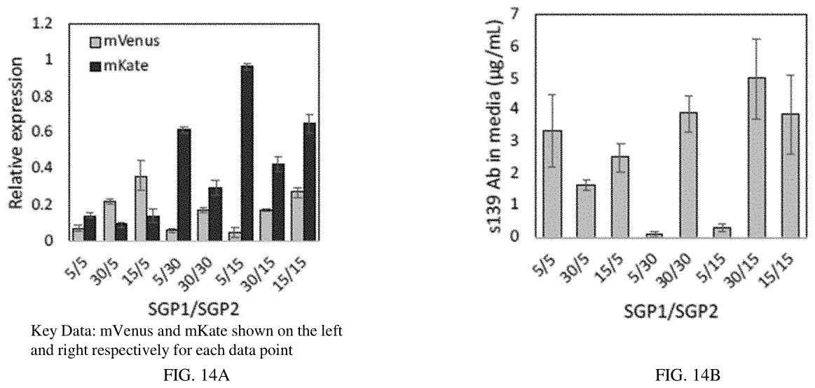

Constitutive Expression of IL-4 and IL-10 and ScFvs Against IL-6 or IL-6R. IL-4 and IL-10 and scFv against IL-6 or IL-6R are co-expressed from DREP to attenuate hyperinflammation. Assays are performed to quantify expression by ELISA as in Example 2. IL-4 and IL 10 secretion is quantified by polarization assay using macrophages. Binding affinity by secreted scFvs to IL-6 and IL-6R is quantified. Amplified levels of expression are achieved using DREP delivered either as a single transcriptional unit encoded on HSV-1 or split into two lentivirus constructs for PDMSC engineering (FIG. 13C). To enhance mAb production, subgenomic promoters that optimize HC:LC ratios are used (FIG. 14).

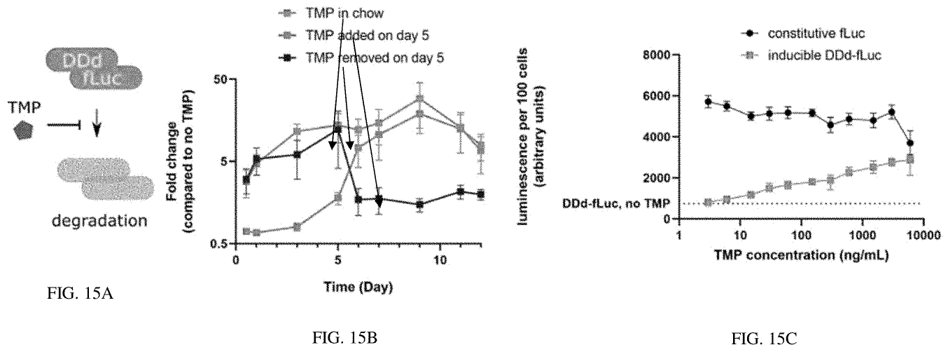

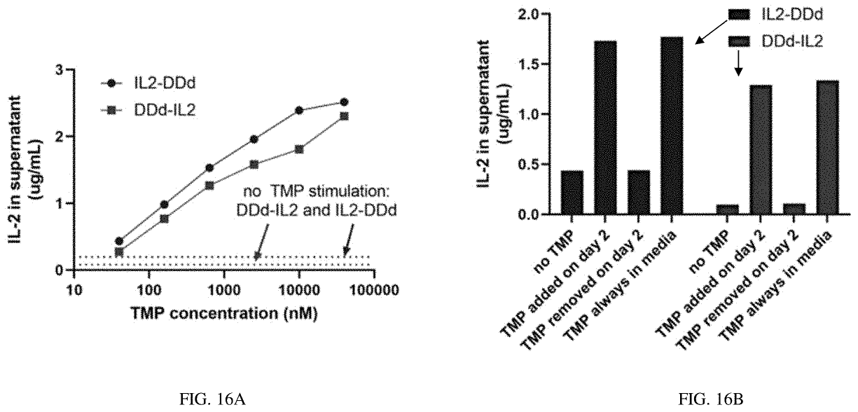

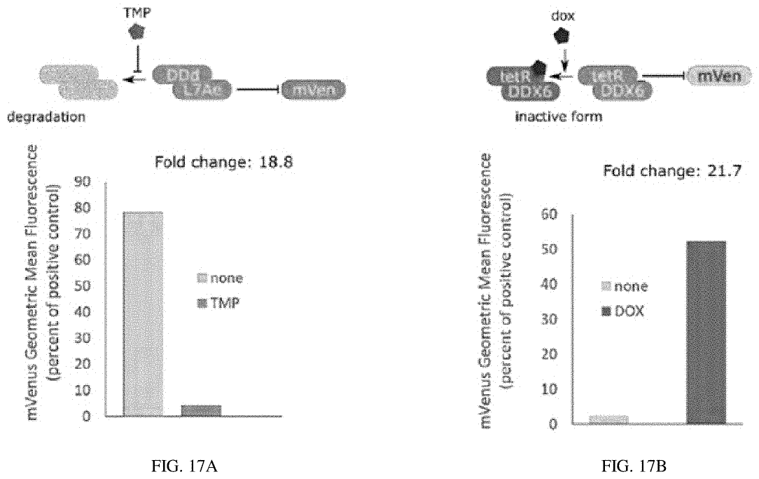

Small Molecule Regulation of Protein Abundance. To improve safety, the abundance of expressed therapeutic proteins is regulated through FDA approved physician-administered small molecule drugs (open loop circuits). Because DREP-based circuits result in high levels of mRNA even when a low level of original gene is transcribed, translational and posttranslational regulation is preferred. Regulation at the post-translational level is realized via regulation of protein stability through a small molecule drug, including E. coli derived dihydrofolate reductase (44) (ecDHFR, also labeled DDd) that binds trimethoprim (TMP), the human estrogen receptor lipid-binding domain (45) (ER-LBD, also labeled DDe) that binds 4-hydroxy-tamoxifen (4-OHT) or the human FKBP-12 derived mutant (DDf) that binds rapamycin analogue Shield-1 (46). 4-OHT is the active metabolite of the FDA approved drug tamoxifen (47) and TMP (48) is an FDA approved antibiotic. Both drugs can be used in human patients, and allow regulation of protein stability at doses lower than their effective dose for prescription treatment. By fusing DDd to a firefly luciferase reporter (FIG. 15A) encoded on self-replicating RNA, it was shown that when TMP is present in the diet of mice injected intramuscularly with lipid nanoparticle encapsulated engineered RNA, luciferase is expressed at much higher levels than in absence of TMP (FIG. 15B). In vitro results in BHK-21 cells demonstrate a dose response curve (FIG. 15C) in the range that corresponds to concentrations typically found in serum of patients treated with TMP (48). It has also been shown that secretion of a DDd-fused cytokine IL-2 is dose dependent on TMP in BHK-21 cells (FIG. 16A) and can be switched on and off by the addition or removal of TMP in cell media (FIG. 16B). A translational regulation mechanism has been developed that does not modify the target gene by using small molecule regulated RNA-binding proteins that act as translational inhibitors (49). Specifically, archaebacterial protein L7Ae was fused to DDd to make it respond to TMP and an L7Ae-binding sequence (kink-turn motif) was encoded in the RNA upstream of a reporter protein. In the presence of TMP, L7Ae is stabilized, binds its target RNA and prevents translation of reporter protein, resulting in an OFF switch (FIG. 17A). An ON switch using DDX6 transcriptional repressor fused to the bacterial transcription factor tetR has also been demonstrated, which also functions as an RNA binding protein and is regulated by FDA approved antibiotic doxycycline (Dox) (FIG. 17B).

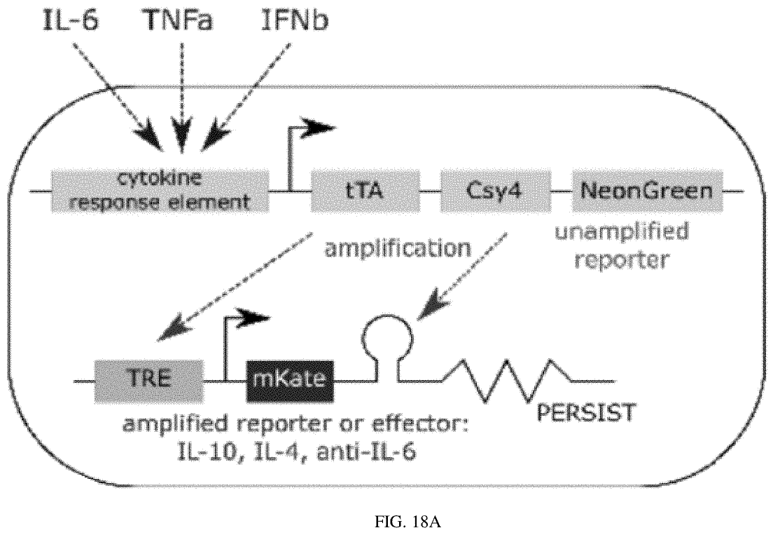

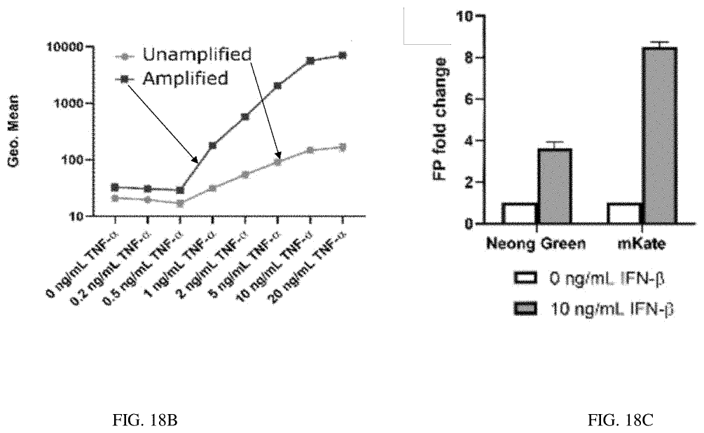

Feedback Regulated Closed Loop Control to Sense IL-6 and Type I Interferon. In the closed loop design, anti-inflammatory proteins are secreted by SpongeBots in response to inflammation without external intervention. This allows automated tuning of immunosuppression to reduce unwanted immune modulation and side effects. For this, mammalian signaling pathways are rewired based on naturally expressed surface receptors and payload therapeutic genes are placed under control of inflammation inducible promoters. Binding of CRS-relevant IL-6 to IL-6R leads to activation of several signaling pathways, such as JAK/STAT (50). The IL-6 receptor signaling pathway is rewired to a synthetic JAK/STAT promoter driving scFv against overt IL-6. The IFNα/IFNβ signaling pathway is also rewired by synthetic IFNAR1/2 and an interferon sensitive response element (ISRE) (51) promoter driving IL-4 and IL-10 expression. Patients with high levels of IL-6 automatically express and secrete scFv against IL-6 to locally attenuate hyperinflammation. With low level of type I interferon, which is generally impaired in early stages of severe COVID-19 patients (52), IL-4 and IL-10 are not expressed and do not immunosuppress. In contrast, at late stages, abundant interferon appears and IL-4/IL-10 is induced to attenuate hyperinflammation as needed. A novel amplified response to activation of immune elements by has been developed by encoding Csy4 protease downstream of the promotor. Separately, a payload gene is encoded with Csy4 recognition sequence and PERSIST (53) inhibitor. When Csy4 is expressed, payload mRNA is cleaved, resulting in increased expression. The system amplifies TNFα response by two orders of magnitude (FIG. 18B). This genetic circuit is used as an alternative method to sense IFNβ (FIG. 18C) and IL-6 to express IL-4, IL-10 and antibodies against IL-6 to attenuate hyperinflammation in patients with severe COVID-19 situations.

Validation of Safety and Efficacy of SpongeBot Immuno-suppression in vivo for SARS-CoV-2. Human ACE2 transgenic mice are infected by sublethal SARS-CoV-2 dose and intravenously and intranasally administrated SpongeBot, mock vehicle, or PBS. Mice are bled and analyzed by Luminex to determine cytokine and chemokine levels in serum. The analysis is performed on days 0, 1, 3, and 7 post administration using established mouse serum Luminex protocols. Meanwhile, mice survival along with CT scans and routine blood analysis is quantified to determine Attenuator benefit.

Alternative Methods. In some embodiments, SpongeBot cells are engineered to produce TGFβ and/or PGE2. In some embodiments, SpongeBot cells are engineered to produce IL-13, nAB against IL-1, TNFα, IFN-γ, and/or IL12. In some embodiments, full-length antibody against IL-6 or IL-6R is used. Antibody expression from DREP has been optimized by modulating SGP strength (FIG. 17). In some embodiments, for creating OFF switches, tightly controlled expression of hBAX apoptosis inducing factor using doxycycline can be used to trigger SpongeBot cell death. Several other options for gene regulation in mammalian cells and animal models have been described and are available including the tet-ON and RheoSwitch systems. Other options, which are analogous to the TetR system, are transcription factors from bacterial antibiotic-resistance genes that were converted to mammalian transcription activators through the fusion with the transcription activating domain VP16 (e.g., pristinamycin and erythromycin).

Outcomes and Metrics. This study develops a version of SpongeBot that removes viral particles and decreases inflammation in infected areas. Cells producing 0.5-1.0 μg IL-4, IL-10, anti-IL-6, and anti-IL-6R/10⁶ cells/day in vitro are engineered. In vivo, the introduction of SpongeBot cells results in levels of 0.5 ng/mL of the same cytokines and antibodies, reducing inflammation markers such as IL-6, TNFα and IFN-γ by 5-10 fold. The levels of cytokines (both inflammatory and anti-inflammatory) are evaluated in animal models infected with SARS-CoV-2. For small molecule-regulated circuits, 20-30 fold regulation is achieved in vitro and 5-15 fold difference between production of cytokines is achieved in the presence and absence of small molecule inducer in vivo. The inducible circuits are tested in the same animal models.

Finally, feedback circuits with 10-100 fold induction of immunomodulator expression upon activation with pro-inflammatory cytokines in vitro and resulting in similar inflammation reduction as small molecule-regulated circuits in vivo are generated. Cytokine expression kinetics and inflammation attenuation measurements are used in combination with viral destruction kinetics measured in Example 2 to determine expected dosage and treatment schedule.

### Example 5: SpongeBot Technology

For reduction of viral load. Unlike a vaccine, the SpongeBot genetic platform is designed to sequester and destroy SARS-CoV-2 even if infection has already taken place. The human disease response can be divided into two phases. Initially, the innate immune system takes a generalized approach attempting to destroy invaders. There is an approximate two-week gap while the second phase, the adaptive immune response, comes online. The adaptive response is more specific and more powerful and is generally necessary to defeat a viral infection. Unfortunately, death or organ damage can take place during this gap period. SpongeBot administration early in the disease process vastly reduces viral load (the amount of virus in the body) during this two-week gap, thus preventing serious infection.

For mitigation of hyperinflammation, virus prophylaxis and viral immunity.

SARS-CoV-2 causes a hyper-inflammatory response by the body's immune system.

Hyperinflammation is responsible for pneumonia/ARDS-like symptoms, which consist of secretions accumulating in the lungs' alveoli, where gas exchange takes place—preventing effective oxygenation of blood and depriving organs of the oxygen they need. The virus also causes damage in blood vessels and in organs like the heart and kidneys. It is the hyperinflammatory state in the lungs and organ damage, which leads to death. SpongeBot not only reduces viral load, but in some embodiments, also secretes anti-inflammatory chemicals (immunosuppressive cytokines and antibodies) that powerfully reduce inflammation, preventing death and organ damage. SpongeBot can also be administered prophylactically as a vaccine—preventing infection; this is particularly valuable to healthcare workers and front-line responders who are vital to infrastructure and often in intimate contact with people infected by the virus.

SpongeBot library. The SpongeBot platform includes a library of RNA sequences anticipated as mutations likely to occur in the SARS-CoV-2 viral RNA. Rapid mutation of RNA is a common characteristic of RNA viruses like SARS-CoV-2. The library contains viral RNA variations based on studies of preserved RNA sequences across the large SARS-corona family of viruses and predictions of how these might mutate. Unlike any extant medications or vaccines, the library allows SpongeBots to be rapidly programmed to address new viral mutations; a SpongeBot tuned to a new viral variation can be produced in 12 weeks or less in comparison to vaccines, which are not therapeutic and take between nine and 12 months to produce. SpongeBots' rapid response capabilities to new viral strains are critical in preventing the kind of human and economic devastation being experienced with the SARS-CoV-2 pandemic. Further, SpongeBot technology can be rapidly expanded to address other, non-SARS-corona viral threats.

### Example 6. SpongeBot cells Protect Target Cells from Infection by a Virus

HSV-1 was used as a model virus to safely study the ability to SpongeBot cells to protect target cells from infection by a virus. HSV-1 is a single molecule, linear dsDNA virus. The HSV-1 genome has at least 74 ORFs with immediate early genes (mostly transcriptional regulators), early genes (mostly DNA replication and some envelope components), and late genes (most structural proteins). HSV-1 infectivity correlates with Nectin-1 expression (FIG. 7).

Targeting HSV-1 genome. The HSV-1 genome was targeted using Cas9 and gRNAs. The gRNA selection criteria were as follows: (i) length 20 bp; (ii) PAM=NGG (G in position 22 and 23); (iii) starts with G for best expression with U6 promoter; (iv) has A or T in position 17; (iv) is in an important gene; (v) is close to the 5′ end of gene; (vi) does not occur in human genome; (vii) avoids low complexity. gRNAs targeting UL48 and ICP4 were designed and tested.

Attraction and destruction of HSV-1 viral particles. Mouse melanoma (Ym) cells that express high levels of HSV-1 entry receptor (Nectin-1) were used as target cells. The base for the therapeutic SpongeBot cells was a different type of mouse melanoma (B16F10) cell. The B16F10 cells were genetically engineered to express only a blue fluorescent protein (without attractor or destroyer), to express green fluorescent protein, Nectin-1 and CRISPR/Cas9 with an gRNA targeting the HSV-1 genome (attractor+functional destroyer), or to express green fluorescent protein, Nectin-1 and CRISPR/Cas9 with nonfunctional gRNA (attractor+nonfunctional destroyer).

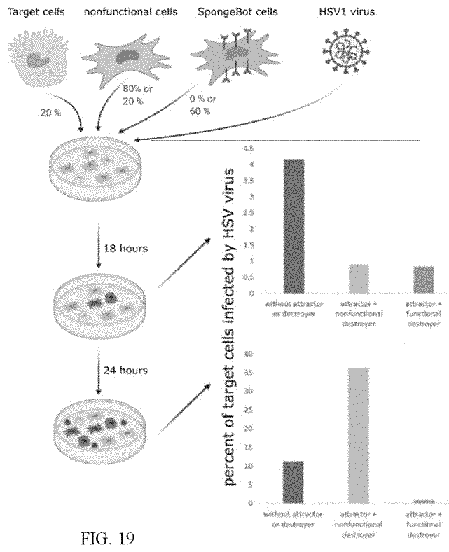

These cells were mixed at different ratios, with target Ym cells always comprising 20% of the total population and the remaining 80% cells comprising a mix of “blue” nonfunctional B16F10 cells without attractor or destroyer and “green” SpongeBot B16F10 cells with attractor and either functional or nonfunctional destroyer. The cells were infected with HSV-1 encoding a red fluorescent protein at multiplicity of infection 0.25 and then the number of target cells expressing the red fluorescent protein was counted after 18 and 42 hours. In the initial 18 hours, the virus enters the target and susceptible SpongeBot cells and expresses the red fluorescent protein. In the following 24 hours, the virus uses the target and SpongeBot cells to generate new viral particles and infects more cells. For the SpongeBot cells that were expressing a functional destroyer, the virus was unable to expand and infect more cells, resulting in the observed low percentage of infected cells on the second day (FIG. 19). Thus, genetically engineered SpongeBot cells protect target cells from infection by a virus.

### Example 7. Targeted Sequence Destruction of SARS-CoV-2

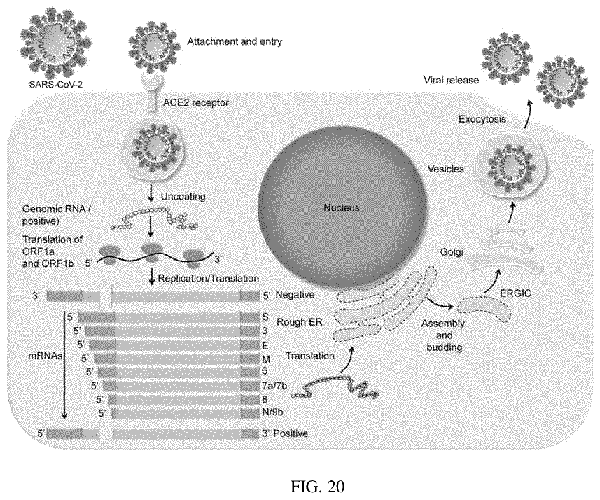

The lifecycle of SARS-CoV-2 is shown in FIG. 20. The SARS-CoV-2 genome is greater than 30 kbp and undergoes discontinuous subgenomic RNA replication. The subgenomes are 5′ and 3′ co-terminal with genomic RNA. 5′ and 3′ sequences are conserved, exposed, and on all viral RNA.

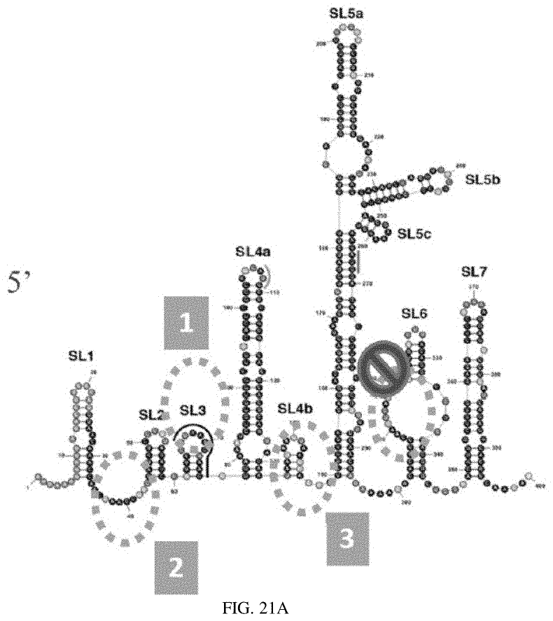

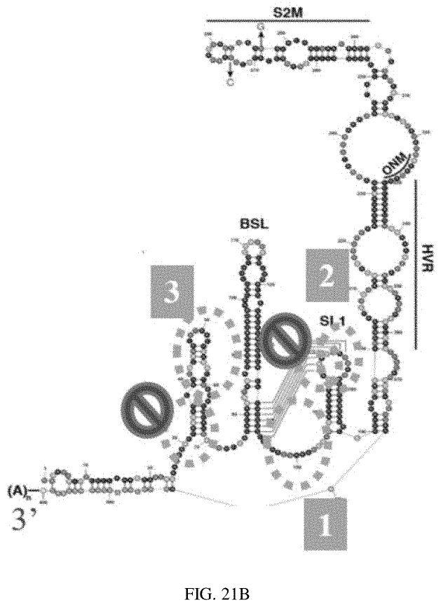

When selecting targets for SARS-CoV-2 destruction, the following criteria were followed: 1) prefer single stranded regions, 2) use multiple targets to prevent evolutionary escape, 3) target both + and − strand, with a preference for the + strand, and 4) remove homology with the human transcriptome (FIG. 21).

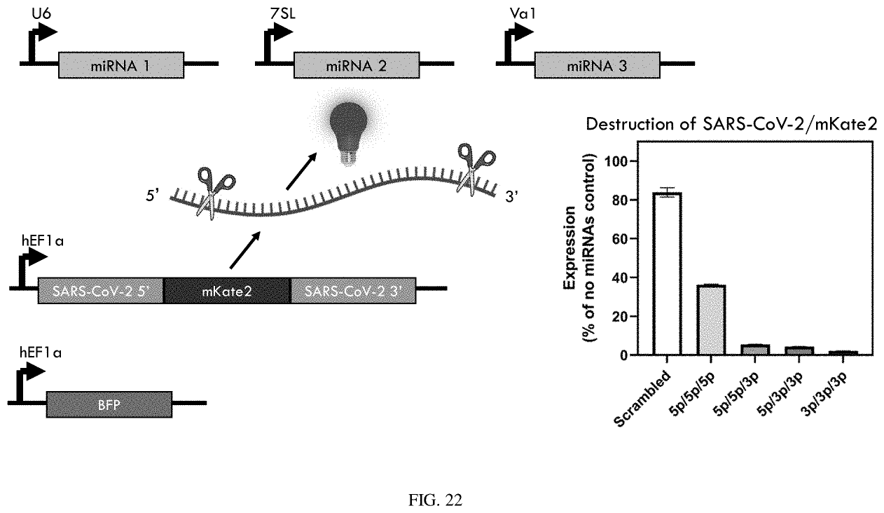

For testing the miRNAs targeting the SAR-CoV-2 genome, a red fluorescent protein (mKate2) has been engineered with about 350nt from the 5′ and 350 nt from the 3′ of the SARS-CoV-2 genome. miRNAs target six different positions on SARS-CoV-2 genome. On the 5′ of SARS-CoV-2 genome, the miRNAs target the positions 18, 48, 125. On the 3′ of SARS-CoV-2 genome, the miRNAs target the positions 29512, 29601, 29623. Four groups of three miRNAs were used in each sample to downregulate the engineered red fluorescent protein. Destruction of the SARS-CoV2-2/mKate2 construct by the miRNAs is shown in FIG. 22. Without wishing to be bound by theory, stronger downregulation with miRNAs targeting the 3′ region is expected because on the 5′, the ribosome can displace the RISC complex.

### REFERENCES

1. Yu, X. et al. SARS-CoV-2 viral load in sputum correlates with risk of COVID-19 progression. Crit Care 24, 170, doi:10.1186/s13054-020-02893-8 (2020).
2. Ullah, I., Subbarao, R. B. & Rho, G. J. Human mesenchymal stem cells—current trends and future prospective. Biosci Rep 35, doi:10.1042/BSR20150025 (2015).
3. Rustad, K. C. & Gurtner, G. C. Mesenchymal Stem Cells Home to Sites of Injury and Inflammation. Adv Wound Care (New Rochelle) 1, 147-152, doi:10.1089/wound.2011.0314 (2012).
4. Ullah, M., Liu, D. D. & Thakor, A. S. Mesenchymal Stromal Cell Homing: Mechanisms and Strategies for Improvement. iScience 15, 421-438, doi:10.1016/j.isci.2019.05.004 (2019).
5. Squillaro, T., Peluso, G. & Galderisi, U. Clinical Trials With Mesenchymal Stem Cells: An Update. Cell Transplant 25, 829-848, doi:10.3727/096368915X689622 (2016).
6. Brooke, G. et al. Manufacturing of human placenta-derived mesenchymal stem cells for clinical trials. Br J Haematol 144, 571-579, doi:10.1111/j.1365-2141.2008.07492.x (2009).
7. Li, W. et al. Angiotensin-converting enzyme 2 is a functional receptor for the SARS coronavirus. Nature 426, 450-454, doi:10.1038/nature02145 (2003).
8. Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
9. Luzina, I. G. et al. Regulation of inflammation by interleukin-4: a review of “alternatives”. J Leukoc Biol 92, 753-764, doi:10.1189/j1b.0412214 (2012).
10. Saraiva, M. & O'Garra, A. The regulation of IL-10 production by immune cells. Nat Rev Immunol 10, 170-181, doi:10.1038/nri2711 (2010).
11. Mihara, M., Hashizume, M., Yoshida, H., Suzuki, M. & Shiina, M. IL-6/IL-6 receptor system and its role in physiological and pathological conditions. Clin Sci (Lond) 122, 143-159, doi:10.1042/C520110340 (2012).
12. Wu, M. et al. Comparison of the Biological Characteristics of Mesenchymal Stem Cells Derived from the Human Placenta and Umbilical Cord. Sci Rep 8, 5014, doi:10.1038/s41598-018-23396-1 (2018).
13. Argonaut. doi:www.argonautms.com/contract-manufacturing/?gclid=CjOKCQjwnv71BRCOARIsAllcxW9Ftn-_eyM0V13kBnMj1j1k4oq49eMoKQ6ic19-Yas-J6WICLHOqBLgaAjyREALw_wcB (2020).
14. Hodgkinson, C. P., Gomez, J. A., Mirotsou, M. & Dzau, V. J. Genetic engineering of mesenchymal stem cells and its application in human disease therapy. Hum Gene Ther 21, 1513-1526, doi:10.1089/hum.2010.165 (2010).
15. Wei, W., Huang, Y., Li, D., Gou, H. F. & Wang, W. Improved therapeutic potential of MSCs by genetic modification. Gene Ther 25, 538-547, doi:10.1038/s41434-018-0041-8 (2018).
16. von Einem, J. C. et al. Treatment of advanced gastrointestinal cancer with genetically modified autologous mesenchymal stem cells—TREAT-ME-1— a phase I, first in human, first in class trial. Oncotarget 8, 80156-80166, doi:10.18632/oncotarget.20964 (2017).
17. Stuckey, D. W. & Shah, K. Stem cell-based therapies for cancer treatment: separating hope from hype. Nat Rev Cancer 14, 683-691, doi:10.1038/nrc3798 (2014).
18. Fakiruddin, K. S., Ghazalli, N., Lim, M. N., Zakaria, Z. & Abdullah, S. Mesenchymal Stem Cell Expressing TRAIL as Targeted Therapy against Sensitised Tumour. Int J Mol Sci 19, doi:10.3390/ijms19082188 (2018).
19. McCarthy, S. D. et al. Nebulized Mesenchymal Stem Cell Derived Conditioned Medium Retains Antibacterial Properties Against Clinical Pathogen Isolates. J Aerosol Med Pulm Drug Deliv, doi:10.1089/jamp.2019.1542 (2019).
20. Aver′yanov, A. V. et al. Survival of Mesenchymal Stem Cells in Different Methods of Nebulization. Bull Exp Biol Med 164, 576-578, doi:10.1007/s10517-018-4034-9 (2018).
21. Kraitchman, D. L. et al. Dynamic imaging of allogeneic mesenchymal stem cells trafficking to myocardial infarction. Circulation 112, 1451-1461, doi:10.1161/CIRCULATIONAHA.105.537480 (2005).
22. Xie, Z., Wroblewska, L., Prochazka, L., Weiss, R. & Benenson, Y. Multi-input RNAi-based logic circuit for identification of specific cancer cells. Science 333, 1307-1311, doi:10.1126/science.1205527 (2011).
23. Gam, J. J., Babb, J. & Weiss, R. A mixed antagonistic/synergistic miRNA repression model enables accurate predictions of multi-input miRNA sensor activity. Nat Commun 9, 2430, doi:10.1038/s41467-018-04575-0 (2018).
24. Yingzhong Li, Z. S. W. Z., Xinfu Zhang, Noor Momin Chengxiang Zhang, K. Dane Wittrup, Yizhou Dong, Darrell J. Irvine, Ron Weiss. Multifunctional oncolytic nanoparticles carrying therapeutic self-replicating RNA eliminate established tumors and prime systemic immunity. Nature Cancer, doi:doi.org/10.1038/s43018-020-0095-6 (2020).
25. Li, T. S. et al. Direct comparison of different stem cell types and subpopulations reveals superior paracrine potency and myocardial repair efficacy with cardiosphere-derived cells. J Am Coll Cardiol 59, 942-953, doi:10.1016/j.jacc.2011.11.029 (2012).
26. Bao, L. et al. The pathogenicity of SARS-CoV-2 in hACE2 transgenic mice. Nature, doi:10.1038/s41586-020-2312-y (2020).
27. Taconic. (2020).
28. Carette, J. E. et al. Ebola virus entry requires the cholesterol transporter Niemann-Pick Cl. Nature 477, 340-343, doi:10.1038/nature10348 (2011).
29. Hasan, S. S. et al. Cryo-EM Structures of Eastern Equine Encephalitis Virus Reveal Mechanisms of Virus Disassembly and Antibody Neutralization. Cell Rep 25, 3136-3147 e3135, doi:10.1016/j.celrep.2018.11.067 (2018).
30. Dou, D., Revol, R., Ostbye, H., Wang, H. & Daniels, R. Influenza A Virus Cell Entry, Replication, Virion Assembly and Movement. Front Immunol 9, 1581, doi:10.3389/fimmu.2018.01581 (2018).
31. Tirado, S. M. & Yoon, K. J. Antibody-dependent enhancement of virus infection and disease. Viral Immunol 16, 69-86, doi:10.1089/088282403763635465 (2003).
32. Shibuya, A. et al. Fc alpha/mu receptor mediates endocytosis of IgM-coated microbes. Nat Immunol 1, 441-446, doi:10.1038/80886 (2000).
33. Chan, M. et al. Generation and Characterization of a Mouse-Adapted Makona Variant of Ebola Virus. Viruses 11, doi:10.3390/v11110987 (2019).
34. de Wit, E., Kawaoka, Y., de Jong, M. D. & Fouchier, R. A. Pathogenicity of highly pathogenic avian influenza virus in mammals. Vaccine 26 Suppl 4, D54-58, doi:10.1016/j.vaccine.2008.07.072 (2008).
35. Honnold, S. P. et al. Eastern equine encephalitis virus in mice II: pathogenesis is dependent on route of exposure. Virol J 12, 154, doi:10.1186/s12985-015-0385-2 (2015).
36. Starr, T. N. et al. Deep Mutational Scanning of SARS-CoV-2 Receptor Binding Domain Reveals Constraints on Folding and ACE2 Binding. Cell 182, 1295-1310 e1220, doi:10.1016/j.cell.2020.08.012 (2020).
37. Peck, K. M. & Lauring, A. S. Complexities of Viral Mutation Rates. J Virol 92, doi:10.1128/JVI.01031-17 (2018).
38. Tom B. Brown, B. M., Nick Ryder, Melanie Subbiah, Jared Kaplan, Prafulla Dhariwal, Arvind Neelakantan, Pranav Shyam, Girish Sastry, Amanda Askell, Sandhini Agarwal, Ariel Herbert-Voss, Gretchen Krueger, Tom Henighan, Rewon Child, Aditya Ramesh, Daniel M. Ziegler, Jeffrey Wu, Clemens Winter, Christopher Hesse, Mark Chen, Eric Sigler, Mateusz Litwin, Scott Gray, Benjamin Chess, Jack Clark, Christopher Berner, Sam McCandlish, Alec Radford, Ilya Sutskever, Dario Amodei. Language Models are Few-Shot Learners. arXiv.org>cs>Computation and Language, doi:arXiv:2005.14165 [cs.CL] (2020).
39. Lasso, G. et al. A Structure-Informed Atlas of Human-Virus Interactions. Cell 178, 1526-1541 e1516, doi:10.1016/j.cell.2019.08.005 (2019).
40. Madeira, F. et al. The EMBL-EBI search and sequence analysis tools APIs in 2019. Nucleic Acids Res 47, W636-W641, doi:10.1093/nar/gkz268 (2019).
41. Bausch-Fluck, D. et al. A mass spectrometric-derived cell surface protein atlas. PLoS One 10, e0121314, doi:10.1371/journal.pone.0121314 (2015).
42. de Wit, E., van Doremalen, N., Falzarano, D. & Munster, V. J. SARS and MERS: recent insights into emerging coronaviruses. Nat Rev Microbiol 14, 523-534, doi:10.1038/nrmicro.2016.81 (2016).
43. Zhang, J. M. & An, J. Cytokines, inflammation, and pain. Int Anesthesiol Clin 45, 27-37, doi:10.1097/AIA.0b013e318034194e (2007).
44. Iwamoto, M., Bjorklund, T., Lundberg, C., Kirik, D. & Wandless, T. J. A general chemical method to regulate protein stability in the mammalian central nervous system. Chem Biol 17, 981-988, doi:10.1016/j.chembiol.2010.07.009 (2010).
45. Miyazaki, Y., Imoto, H., Chen, L. C. & Wandless, T. J. Destabilizing domains derived from the human estrogen receptor. J Am Chem Soc 134, 3942-3945, doi:10.1021/ja209933r (2012).
46. Banaszynski, L. A., Chen, L. C., Maynard-Smith, L. A., Ooi, A. G. & Wandless, T. J. A rapid, reversible, and tunable method to regulate protein function in living cells using synthetic small molecules. Cell 126, 995-1004, doi:10.1016/j.cell.2006.07.025 (2006).
47. Pharmaceuticals, R. SOLTAMOX® (tamoxifen citrate) oral solution. doi:soltamox.com.
48. USA, T. P. TMP, TRIMETHOPRIM TABLETS, USP. doi:www.tevausa.com.
49. Wagner, T. E. et al. Small-molecule-based regulation of RNA-delivered circuits in mammalian cells. Nat Chem Biol 14, 1043-1050, doi:10.1038/s41589-018-0146-9 (2018).
50. Wang, L. et al. IL-17 can promote tumor growth through an IL-6-Stat3 signaling pathway. J Exp Med 206, 1457-1464, doi:10.1084/jem.20090207 (2009).
51. Promega. pGL4.45[luc2P/ISRE/Hygro] Vector.doi:www.promega.com/resources/protocols/product-information-sheets/a/pg14-45-vector-protocol/.
52. Hadjadj, J. et al. Impaired type I interferon activity and inflammatory responses in severe COVID-19 patients. Science 369, 718-724, doi:10.1126/science.abc6027 (2020).
53. Breanna DiAndreth, N. W., Eileen Hu, Sebastian Palacios, Ron Weiss. PERSIST: A programmable RNA regulation platform using CRISPR endoRNases. bioRxiv, doi:doi.org/10.1101/2019.12.15.867150 (2019).

### OTHER EMBODIMENTS

In some embodiments, the present disclosure provides:

1. A genetically-engineered immune cell for treating COVID-19, comprising a
(i) a SARS-CoV2 Super-Attractor, wherein the Super-Attractor over-expresses receptor complexes (optionally including ACE2 or TMPRSS2) that bind the virus and promote viral entry into the genetically-engineered immune cell cytoplasm;
(ii) a SARS-CoV2 RNA destroyer, wherein the destroyer destroys viral RNA when the virus enters the genetically-engineered immune cell, prior to viral activation and replication; and
(iii) a Cytokine Release Storm (CRS) Attenuator, wherein the genetically-engineered immune cell attenuates Cytokine Release Storm, thus preventing dangerous inflammation in the lung.
2. A gene therapy method of treating a patient with COVID 19, comprising administering to a patient (via inhalation or injection) HSV-1 amplicons that contain the genetic program of claim 1 to perform the Super-Attractor, RNA destroyer and CRS attenuator functions.
3. A cell-based therapy method of treating a patient with COVID19, comprising creating the genetically-engineered immune cell of claim 1 ex-vivo via genetic engineering of Placental Derived Mesenchymal Stem Cells (PDMSCs), and administering the PDMSCs to a patient.
4. A library of HSV-1 Amplicons to be incorporated into a genetically engineered immune cell to be used for future viral pandemic threats, wherein the library comprises expression of human cell surface receptors that could be targeted by viruses and conserved viral DNA/RNA sequences.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered genetic system comprising (i) one or more nucleotide sequences encoding one or more components that bind a virus; and one or more of: (ii) one or more nucleotide sequences encoding one or more inhibitory oligonucleotides targeting the viral genome; and (iii) one or more nucleotide sequences encoding one or more anti-inflammatory molecules.

2. The engineered genetic system of claim 1, wherein the virus is a DNA virus or an RNA virus.

3. (canceled)

4. The engineered genetic system of claim 1, wherein the virus is selected from the group consisting of: Adenoviridae, Coronaviridae, Flaviviridae, Filoviridae, Herpesviridae, Hepadnaviridae, Orthomyxoviridae, Paramyxoviridae, Papillomaviridae, Picornaviridae, Polyomaviridae, Poxviridae, Retroviridae, Rhabdoviridae, and Togaviridae.

5. The engineered genetic system of claim 4, wherein the virus is selected from the group consisting of: severe acute respiratory syndrome coronavirus (SARS-CoV), SARS-CoV-2, Middle East respiratory syndrome coronavirus (MERS-CoV), influenza virus, dengue virus, zika virus, ebola virus, variola virus, rabies virus, measles virus, human immunodeficiency virus (HIV), Venezuelan equine encephalitis virus (VEEV), Eastern equine encephalitis virus (EEEV), and herpes simplex virus type 1 (HSV-1).

6. The engineered genetic system of claim 1, wherein the component that binds the virus is a viral receptor or accessory protein, optionally wherein the viral receptor or accessory protein is ACE2, TMPRSS2, cathepsin B, cathepsin L, nectin-1, DPP4, CD4, LDLR, low density LDLRAD3, CCR5, CXCR4, SR-B1, CD81, claudin-1, occludin, CAR, NPC1, TIM-1, DC-SIGN, L-SIGN, hMGL, TYRO-3, AXL, MER, JAM-A, αvβ3, αvβ5, Gas6, CD21, AChR, EGFR, EFNB2, CD46, SLAMF1, nectin-4, or a CAM.

7. (canceled)

8. The engineered genetic system of claim 1, wherein the component that binds the virus is an antibody, optionally wherein the antibody is an IgM antibody.

9. (canceled)

10. The engineered genetic system of claim 1, wherein the one or more inhibitory oligonucleotides target the RNA-dependent RNA polymerase of the virus, the 5′ UTR region of the viral genome, and/or the 3′ UTR region of the viral genome.

11. The engineered genetic system of claim 1, wherein at least one of the one or more inhibitory oligonucleotides is (i) an interfering RNA, optionally wherein the interfering RNA is an miRNA; or (ii) a clustered regularly interspaced short palindromic repeats (CRISPR) guide RNA (gRNA), optionally a Cas9/gRNA or a Cas13/gRNA.

12. (canceled)

13. The engineered genetic system of claim 11, wherein the system comprises a CRISPR gRNA and further comprises a nucleotide sequence encoding a Cas protein, optionally wherein the Cas protein is Cas9 or Cas13.

14. The engineered genetic system of claim 1, wherein the anti-inflammatory molecule is (i) a cytokine, optionally wherein the cytokine is IL-4, IL-10, IL-11, IL-13, IL-1Rα, TGFβ, or PGE2, or (ii) an antibody that binds an inflammatory molecule, optionally wherein the inflammatory molecule is IL-6, IL-6R, IL-1, IL-12, IL-18, IFNγ, GM-CSF, or TNF-α.

15. (canceled)

16. The engineered genetic system of claim 1, wherein one or more of (i), (ii), and (iii) are present in one or more vectors.

17. The engineered genetic system of claim 16, wherein the one or more vectors are viral vectors, optionally wherein the viral vectors are adeno associated virus vectors, Sendai virus vectors, lentiviral vectors, γ-retroviral vectors, or HSV-1 amplicons.

18. (canceled)

19. The engineered genetic system of claim 1, wherein one or more of (i), (ii), and (iii) are present in lipid nanoparticles.

20. The engineered genetic system of claim 1, wherein a nucleic acid molecule comprising the nucleotide sequences of (i), a nucleic acid molecule comprising the nucleotide sequences of (ii), or a nucleic acid molecule comprising the nucleotide sequences of (iii) encodes a self-amplifying RNA.

21. An engineered cell comprising the engineered genetic system of claim 1.

22. The engineered cell of claim 21, wherein the engineered cell is (i) an immune cell, or (ii) a stem cell, optionally wherein the stem cell is a mesenchymal stem cell (MSC).

23. (canceled)

24. A method of treating or preventing a viral infection, reducing viral load, or reducing viral spread in a subject in need thereof, comprising administering to the subject an effective amount of the engineered genetic system of claim 1.

25. A method of treating or preventing a viral infection, reducing viral load, or reducing viral spread in a subject in need thereof, comprising administering to the subject an effective amount of the engineered cell of claim 21.

26.-28. (canceled)

29. A method comprising introducing into, or expressing in, a eukaryotic cell (i) one or more components that bind a virus, and one or more of: (ii) one or more inhibitory nucleic acids targeting the viral genome, and (iii) one or more anti-inflammatory molecules.

30.-32. (canceled)

33. A library of HSV-1 amplicons, comprising a plurality of the genetic engineered system according to claim 1.
